SEQUENCE LISTING (105 sequences)

SEQ ID NO: 1 - protein, 99 aa, Homo sapiens
VHEDSGDEDGEDPDKRISIRASDKRIACDEEFSDSEDEGEGGRRNVADHKKGAKKARIEEDKKETEDKKTDVKEEDKSKDNSGEKTDTKGTKSEQLSNP

SEQ ID NO: 2 - DNA, 9 bp, Artificial Sequence
  Synthetic polynucleotide
ttcaagaga

SEQ ID NO: 3 - DNA, 20 bp, Artificial Sequence
  Synthetic polynucleotide
aattctccgaacgtgtcacg

SEQ ID NO: 4 - DNA, 21 bp, Artificial Sequence
  Synthetic polynucleotide
ggtcgtaggaatgttgctgat

SEQ ID NO: 5 - DNA, 21 bp, Artificial Sequence
  Synthetic polynucleotide
gcacctgtcccaactgtaaag

SEQ ID NO: 6 - DNA, 19 bp, Artificial Sequence
  Synthetic polynucleotide
ggaacagaaggaagaggaa

SEQ ID NO: 7 - DNA, 21 bp, Artificial Sequence
  Synthetic polynucleotide
gccatcaggatttcaagtaat

SEQ ID NO: 8 - DNA, 49 bp, Artificial Sequence
  Synthetic polynucleotide
gatgaaggtgaaggaggccgcagaaacgtggcagaccataagaaaggag

SEQ ID NO: 9 - DNA, 49 bp, Artificial Sequence
  Synthetic polynucleotide
ctcctttcttatggtctgccacgtttctgcggcctccttcaccttcatc

SEQ ID NO: 10 - DNA, 59 bp, Artificial Sequence
  Synthetic polynucleotide
gtacctctcccaccaccttccttgcaattcgggcacgtacaagctaccctccgaagtct

SEQ ID NO: 11 - DNA, 59 bp, Artificial Sequence
  Synthetic polynucleotide
agacttcggagggtagcttgtacgtgcccgaattgcaaggaaggtggtgggagaggtac

SEQ ID NO: 12 - DNA, 20 bp, Artificial Sequence
  Synthetic polynucleotide
ctaccctctcccctgtctcc

SEQ ID NO: 13 - DNA, 20 bp, Artificial Sequence
  Synthetic polynucleotide
gcaaagaaaacaccccattc

SEQ ID NO: 14 - DNA, 18 bp, Artificial Sequence
  Synthetic polynucleotide
agtgtccggcattctgct

SEQ ID NO: 15 - DNA, 18 bp, Artificial Sequence
  Synthetic polynucleotide
ctcgcccacccagactac

SEQ ID NO: 16 - DNA, 20 bp, Artificial Sequence
  Synthetic polynucleotide
tcaatccttgtcccttttgc

SEQ ID NO: 17 - DNA, 20 bp, Artificial Sequence
  Synthetic polynucleotide
caagcaagcacatccacatc

SEQ ID NO: 18 - DNA, 20 bp, Artificial Sequence
  Synthetic polynucleotide
caggaaaggaagaggggaac

SEQ ID NO: 19 - DNA, 20 bp, Artificial Sequence
  Synthetic polynucleotide
agtgagacaaagccctccaa

SEQ ID NO: 20 - DNA, 20 bp, Artificial Sequence
  Synthetic polynucleotide
gctgagatgtggttggcttt

SEQ ID NO: 21 - DNA, 20 bp, Artificial Sequence
  Synthetic polynucleotide
cccccaagcctattctgttt

SEQ ID NO: 22 - DNA, 20 bp, Artificial Sequence
  Synthetic polynucleotide
gtgaatcaggtggggacatc

SEQ ID NO: 23 - DNA, 20 bp, Artificial Sequence
  Synthetic polynucleotide
caacaagaccacaggaagca

SEQ ID NO: 24 - DNA, 20 bp, Artificial Sequence
  Synthetic polynucleotide
tctccatctggctaccaacc

SEQ ID NO: 25 - DNA, 20 bp, Artificial Sequence
  Synthetic polynucleotide
agagggaagacggaaaggag

SEQ ID NO: 26 - DNA, 20 bp, Artificial Sequence
  Synthetic polynucleotide
aagaggggcagagaaagctc

SEQ ID NO: 27 - DNA, 20 bp, Artificial Sequence
  Synthetic polynucleotide
gggacaaacttcctcccttc

SEQ ID NO: 28 - DNA, 20 bp, Artificial Sequence
  Synthetic polynucleotide
tcgctgtgctgctgagttag

SEQ ID NO: 29 - DNA, 20 bp, Artificial Sequence
  Synthetic polynucleotide
cagaaagctcagggattgga

SEQ ID NO: 30 - DNA, 20 bp, Artificial Sequence
  Synthetic polynucleotide
ttcgcctacgtgctcaacta

SEQ ID NO: 31 - DNA, 20 bp, Artificial Sequence
  Synthetic polynucleotide
gtctcgtctatgccccagaa

SEQ ID NO: 32 - DNA, 20 bp, Artificial Sequence
  Synthetic polynucleotide
agcagtacccaaccaccttg

SEQ ID NO: 33 - DNA, 18 bp, Artificial Sequence
  Synthetic polynucleotide
ctccagagccccacgttc

SEQ ID NO: 34 - DNA, 20 bp, Artificial Sequence
  Synthetic polynucleotide
gagctagccatcgagcagat

SEQ ID NO: 35 - DNA, 20 bp, Artificial Sequence
  Synthetic polynucleotide
acctcggtgtcgtagagtcg

SEQ ID NO: 36 - DNA, 20 bp, Artificial Sequence
  Synthetic polynucleotide
cgcccataatctacctttgc

SEQ ID NO: 37 - DNA, 20 bp, Artificial Sequence
  Synthetic polynucleotide
gtgggggaaattccatgata

SEQ ID NO: 38 - DNA, 20 bp, Artificial Sequence
  Synthetic polynucleotide
agaccgcagggattctaggt

SEQ ID NO: 39 - DNA, 21 bp, Artificial Sequence
  Synthetic polynucleotide
agccacagcagaagacaaaag

SEQ ID NO: 40 - DNA, 19 bp, Artificial Sequence
  Synthetic polynucleotide
cctcttcaatgggcaacct

SEQ ID NO: 41 - DNA, 18 bp, Artificial Sequence
  Synthetic polynucleotide
gaatcggtctgccaggtg

SEQ ID NO: 42 - DNA, 20 bp, Artificial Sequence
  Synthetic polynucleotide
agtgggcttcagctcctgta

SEQ ID NO: 43 - DNA, 20 bp, Artificial Sequence
  Synthetic polynucleotide
gccgcgtaggtcttcttatg

SEQ ID NO: 44 - DNA, 20 bp, Artificial Sequence
  Synthetic polynucleotide
aagccgagaggagtgagaca

SEQ ID NO: 45 - DNA, 19 bp, Artificial Sequence
  Synthetic polynucleotide
ccgctcggaagactaggag

SEQ ID NO: 46 - DNA, 20 bp, Artificial Sequence
  Synthetic polynucleotide
tgtgccacaccctaccctat

SEQ ID NO: 47 - DNA, 20 bp, Artificial Sequence
  Synthetic polynucleotide
tgccttgattaatgggttcc

SEQ ID NO: 48 - DNA, 20 bp, Artificial Sequence
  Synthetic polynucleotide
cacattctgtagcccagacg

SEQ ID NO: 49 - DNA, 20 bp, Artificial Sequence
  Synthetic polynucleotide
cagaatctcgggcttctacg

SEQ ID NO: 50 - DNA, 20 bp, Artificial Sequence
  Synthetic polynucleotide
cagtgtgctttctccccttc

SEQ ID NO: 51 - DNA, 20 bp, Artificial Sequence
  Synthetic polynucleotide
agaggtttggggcctgtatt

SEQ ID NO: 52 - DNA, 20 bp, Artificial Sequence
  Synthetic polynucleotide
tgggcctcagtttcctctta

SEQ ID NO: 53 - DNA, 20 bp, Artificial Sequence
  Synthetic polynucleotide
catagccaagagcatcgaca

SEQ ID NO: 54 - DNA, 20 bp, Artificial Sequence
  Synthetic polynucleotide
aacggacagaaggcaaagtg

SEQ ID NO: 55 - DNA, 20 bp, Artificial Sequence
  Synthetic polynucleotide
caaagctcacgggatcaaag

SEQ ID NO: 56 - DNA, 20 bp, Artificial Sequence
  Synthetic polynucleotide
gaagggatgcagccttgtta

SEQ ID NO: 57 - DNA, 20 bp, Artificial Sequence
  Synthetic polynucleotide
ttgagcctttttggttttcc

SEQ ID NO: 58 - DNA, 20 bp, Artificial Sequence
  Synthetic polynucleotide
agagagagagcgagctgcat

SEQ ID NO: 59 - DNA, 20 bp, Artificial Sequence
  Synthetic polynucleotide
ttgaagccagacacagcaac

SEQ ID NO: 60 - DNA, 20 bp, Artificial Sequence
  Synthetic polynucleotide
ccctgacattggtgctcttt

SEQ ID NO: 61 - DNA, 20 bp, Artificial Sequence
  Synthetic polynucleotide
tggttggcaacagtggacta

SEQ ID NO: 62 - DNA, 20 bp, Artificial Sequence
  Synthetic polynucleotide
ctgaagggaactggttcgag

SEQ ID NO: 63 - DNA, 20 bp, Artificial Sequence
  Synthetic polynucleotide
cctgcctgtcctgttcattt

SEQ ID NO: 64 - DNA, 20 bp, Artificial Sequence
  Synthetic polynucleotide
atttcgtcttctgggtgagc

SEQ ID NO: 65 - DNA, 18 bp, Artificial Sequence
  Synthetic polynucleotide
ccttctcagcagggccta

SEQ ID NO: 66 - DNA, 20 bp, Artificial Sequence
  Synthetic polynucleotide
cacttccatcagcagggatt

SEQ ID NO: 67 - DNA, 20 bp, Artificial Sequence
  Synthetic polynucleotide
ggggctgtgtctgctcttta

SEQ ID NO: 68 - DNA, 20 bp, Artificial Sequence
  Synthetic polynucleotide
cctcggtgtctagtggaagg

SEQ ID NO: 69 - DNA, 20 bp, Artificial Sequence
  Synthetic polynucleotide
gcgtttagctaggcggagat

SEQ ID NO: 70 - DNA, 19 bp, Artificial Sequence
  Synthetic polynucleotide
ctgcctccgaatgaatgtg

SEQ ID NO: 71 - DNA, 20 bp, Artificial Sequence
  Synthetic polynucleotide
agaccaacctcggtgacaac

SEQ ID NO: 72 - DNA, 20 bp, Artificial Sequence
  Synthetic polynucleotide
atctcgttctggcagtctgg

SEQ ID NO: 73 - DNA, 20 bp, Artificial Sequence
  Synthetic polynucleotide
acacgaacagaaagccatcc

SEQ ID NO: 74 - DNA, 20 bp, Artificial Sequence
  Synthetic polynucleotide
gacggcattgacgacgaatc

SEQ ID NO: 75 - DNA, 20 bp, Artificial Sequence
  Synthetic polynucleotide
tatggggaatactttcctgg

SEQ ID NO: 76 - DNA, 21 bp, Artificial Sequence
  Synthetic polynucleotide
gcacccacaagacacctatga

SEQ ID NO: 77 - DNA, 20 bp, Artificial Sequence
  Synthetic polynucleotide
cagttgtgtatgacgacagc

SEQ ID NO: 78 - DNA, 20 bp, Artificial Sequence
  Synthetic polynucleotide
ccgaagcttgtcaacaagag

SEQ ID NO: 79 - DNA, 20 bp, Artificial Sequence
  Synthetic polynucleotide
gctgctatcagtgaacggag

SEQ ID NO: 80 - DNA, 20 bp, Artificial Sequence
  Synthetic polynucleotide
tttgaggacccctactcgtc

SEQ ID NO: 81 - DNA, 20 bp, Artificial Sequence
  Synthetic polynucleotide
tcaacgacaccataaggttc

SEQ ID NO: 82 - DNA, 21 bp, Artificial Sequence
  Synthetic polynucleotide
aagactggaaatgttgctcgg

SEQ ID NO: 83 - DNA, 20 bp, Artificial Sequence
  Synthetic polynucleotide
tttgctaaaactggtgaccc

SEQ ID NO: 84 - DNA, 22 bp, Artificial Sequence
  Synthetic polynucleotide
gattgcttcccctagcttctct

SEQ ID NO: 85 - DNA, 20 bp, Artificial Sequence
  Synthetic polynucleotide
atggagggagcatcctttgg

SEQ ID NO: 86 - DNA, 20 bp, Artificial Sequence
  Synthetic polynucleotide
ccccaggtttccgagaaaag

SEQ ID NO: 87 - DNA, 20 bp, Artificial Sequence
  Synthetic polynucleotide
acagccctgtcatgtccagc

SEQ ID NO: 88 - DNA, 22 bp, Artificial Sequence
  Synthetic polynucleotide
tacctaatcattatgccgagga

SEQ ID NO: 89 - DNA, 20 bp, Artificial Sequence
  Synthetic polynucleotide
tcatcagaccccagaaaagg

SEQ ID NO: 90 - DNA, 20 bp, Artificial Sequence
  Synthetic polynucleotide
tgaagcaacctaaccggtcc

SEQ ID NO: 91 - DNA, 20 bp, Artificial Sequence
  Synthetic polynucleotide
tgacagcatagtattttccc

SEQ ID NO: 92 - DNA, 21 bp, Artificial Sequence
  Synthetic polynucleotide
gtctctgggaactgggctaag

SEQ ID NO: 93 - DNA, 20 bp, Artificial Sequence
  Synthetic polynucleotide
agattgtaccttgatggagc

SEQ ID NO: 94 - DNA, 20 bp, Artificial Sequence
  Synthetic polynucleotide
gtgtaccctgaccattcatc

SEQ ID NO: 95 - DNA, 20 bp, Artificial Sequence
  Synthetic polynucleotide
gcagccttgagaagttccac

SEQ ID NO: 96 - DNA, 20 bp, Artificial Sequence
  Synthetic polynucleotide
atgaagccctcgtgtgtctc

SEQ ID NO: 97 - DNA, 20 bp, Artificial Sequence
  Synthetic polynucleotide
ggatgctatacagtggaagc

SEQ ID NO: 98 - DNA, 21 bp, Artificial Sequence
  Synthetic polynucleotide
gctcgccaaggcttttaagaa

SEQ ID NO: 99 - DNA, 20 bp, Artificial Sequence
  Synthetic polynucleotide
aagcggttgggtttggtatg

SEQ ID NO: 100 - DNA, 21 bp, Artificial Sequence
  Synthetic polynucleotide
aggaaatctttaccgccctca

SEQ ID NO: 101 - DNA, 21 bp, Artificial Sequence
  Synthetic polynucleotide
caccgcaatagaaaacaccca

SEQ ID NO: 102 - DNA, 20 bp, Artificial Sequence
  Synthetic polynucleotide
ccaccaatgatggtaaaccc

SEQ ID NO: 103 - DNA, 20 bp, Artificial Sequence
  Synthetic polynucleotide
caaatgtttccagcccagag

SEQ ID NO: 104 - DNA, 20 bp, Artificial Sequence
  Synthetic polynucleotide
gagcaagtctttcagtcctg

SEQ ID NO: 105 - DNA, 20 bp, Artificial Sequence
  Synthetic polynucleotide
gattcggcaggtgagttgtt

(19) United States
(12) Patent Application Publication
(10) Pub. No.: US 2019/0015473 A1
(43) Pub. Date: Jan. 17, 2019

(54) TARGETING THE HDAC2-SP3 COMPLEX TO ENHANCE SYNAPTIC FUNCATION

(71) Applicant: Massachusetts Institute of Technology, Cambridge, MA (US)

(72) Inventors: Li-Huei Tsai, Cambridge, MA (US); Hidekuni Yamakawa, Cambridge, MA (US); Jemmie Cheng, Elmsford, NY (US); Fan Gao, Cambridge, MA (US); Jay Penney, Malden, MA (US)

(73) Assignee: Massachusetts Institute of Technology, Cambridge, MA (US)

(21) Appl. No.: 16/033,635

(22) Filed: Jul. 12, 2018

Related U.S. Application Data
(60) Provisional application No. 62/532,026, filed on Jul. 13, 2017.

(51) Int. Cl. A61K 38/16; A61P 25/28

(52) U.S. Cl. CPC A61K 38/16; A61K 45/06; A61P 25/28

(57) ABSTRACT

The present disclosure provides, in some embodiments, methods for treating a neurodegenerative disease in a subject using a histone deacetylase 2 (HDAC2)/Sp3 inhibitor, which may be a peptide inhibitor comprising the carboxyl-terminus of HDAC2, and related compositions.

### RELATED APPLICATION

This application claims priority under 35 U.S.C. 119(e) to U.S. provisional patent application, U.S. Ser. No. 62/532,026, filed Jul. 13, 2017, which is incorporated herein by reference in its entirety.

### BACKGROUND

Neurodegenerative diseases of the central nervous system are often associated with impaired learning and memory, eventually leading to dementia. The histone deactylase HDAC2, which negatively regulates neuronal plasticity and synaptic gene expression, is upregulated in both Alzheimer's disease (AD) patients and mouse models.

### SUMMARY

The present disclosure is based, at least in part, on the unexpected discoveries that the transcription factor Sp3 (Sp3) mediated recruitment of HDAC2 to the promoters of synaptic plasticity-associated genes and that HDAC2 inhibitors that disrupt that interaction such as peptide inhibitors successfully reduced synaptic and cognitive dysfunction in a mouse model of neurodegeneration.

Accordingly, one aspect of the present disclosure provides a method for treating a neurodegenerative disease in a subject, comprising administering to the subject an effective amount of a histone deacetylase 2 (HDAC2) inhibitor, wherein the HDAC2 inhibitor reduces HDAC2 binding to transcription factor Sp3 (Sp3). In some embodiments, the HDAC2 inhibitor may be an anti-HDAC2 antibody, a small molecule inhibitor, or a peptide inhibitor. The subject to be treated in the methods described herein can be a patient (e.g., a human patient) who has a neurodegenerative disease. In some examples, the neurodegenerative disease is selected from the group consisting of MCI (mild cognitive impairment), post-traumatic stress disorder (PTSD), Alzheimer's Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, vascular dementia, progressive supranuclear palsy or cortical basal degeneration.

In some embodiments, the amount of HDAC2 inhibitor is effective in reducing synaptic dysfunction. Alternatively or in addition, the amount of HDAC2 inhibitor is effective in reducing histone deacetylation. Any of the HDAC2 inhibitors may be administered systemically, e.g., via an enteral route or via a parenteral route. Any of the subjects to be treated by the method described herein may have been administered another therapeutic agent.

In other aspects, the invention is a pharmaceutical composition for treating a neurodegenerative disease in a subject, the composition comprising (i) an effective amount of a histone deacetylase 2 (HDAC2) inhibitor; and (ii) a pharmaceutically acceptable carrier. In some embodiments, the pharmaceutical composition comprises an amount of a HDAC2 inhibitor is effective in reducing HDAC2 binding to transcription factor Sp3 (Sp3).

In yet other aspects, the invention is a peptide inhibitor comprising an amino acid sequence that is at least 80% identical to SEQ ID NO: 1. In some embodiments, the peptide inhibitor is about 25-110 amino acids in length. In other embodiments, the peptide inhibitor comprises an amino acid sequence that is at least 85%, at least 90%, at least 95%, or at least 99% identical to SEQ ID NO: 1. In some embodiments, the peptide inhibitor comprises the amino acid sequence of SEQ ID NO: 1. In some embodiments, the peptide inhibitor consists of the amino acid sequence of SEQ ID NO: 1. In some embodiments, the peptide inhibitor is formulated in a pharmaceutical composition, which further comprises a pharmaceutically acceptable carrier.

The details of one or more embodiments of the invention are set forth in the description below. Other features or advantages of the present invention will be apparent from the following drawings and detailed description of several embodiments, and also from the appended claims.

### BRIEF DESCRIPTION OF DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1E show Sp3 regulates synaptic function and synaptic gene expression. FIG. 1A shows a representative western blot of co-immunoprecipitation of Sp3 with anti-HDAC2 antibody from mouse cortical tissue. FIG. 1B shows representative mEPSC traces (top) and quantifications of mEPSC amplitude and frequency (bottom) from neurons transduced with control shRNA, HDAC2 shRNA or Sp3 shRNA (n=6-12). ** P<0.01, *** P<0.001 (two-tailed Welch's or Student's t-test depending on the result of f-test). FIG. 1C shows representative traces of mEPSC amplitude and frequency in neurons transduced with control shRNA, Sp3 shRNA or shRNA-resistant Sp3 combined with Sp3 shRNA (n=6-8). * P<0.05, ** P<0.01 (Dunnett's test). Values are means±s.e.m. FIG. 1D shows a comparison matrix of differentially expressed genes following HDAC2 shRNA or Sp3 shRNA expression in primary cortical neurons. P-values were calculated using the Fisher's exact test. Genes in black indicate no change in expression, dark grey indicates a decrease in expression, and light grey indicates an increase in expression after treatment with HDAC2 or Sp3 shRNA. HDAC2 and Sp3 shRNA both mediate the decreased expression of Group 1 genes and the increased expression of Group 2 genes. FIG. 1E shows a gene ontology analysis of genes up-regulated by HDAC2 shRNA and Sp3 shRNA using DAVID.

FIGS. 2A-2C show Sp3 knockdown decreases HDAC2 recruitment to target genes. FIG. 2A shows a schematic depiction of neuronal sorting for ChIP experiments. FIG. 2B shows ChIP-qPCR results of HDAC2 (top panel) and Sp3 (bottom panel) at the promoters of potential target genes and control genes identified by RNA-seq in neurons sorted from mouse cortices (n=3). The locations of the amplified regions relative to each genes transcription start site are indicated. FIG. 2C shows ChIP-qPCR results of HDAC2 (top panel) and acetylated histone H4 (bottom panel) at the promoters of the target genes in primary neurons transduced with Sp3 shRNA or control virus (n=3). * P<0.05, ** P<0.01 (Dunnett's test). Values are means±s.e.m.

FIGS. 3A-3E show HDAC2 and Sp3 expression is elevated in AD patients, and anti-correlated with synaptic gene expression. FIG. 3A shows mRNA levels of HDAC2 in postmortem hippocampal CA1 tissue from 13 healthy controls and 10 AD patients. ** P<0.01 (two-tailed Student's t-test). FIG. 3B shows mRNA levels of Sp3 in postmortem hippocampal CA1 tissue from 13 healthy controls and 10 AD patients. ** P<0.01 (two-tailed Student's t-test). FIG. 3C shows gene dendrogram and co-expression modules generated from the dataset of 13 control and 10 AD patients. FIG. 3D shows the correlation matrix of the expression of eigengenes from the identified modules for relationship comparison between modules. Each eigengene is the gene which best represents the standardized expression data for a given module. The module where synaptic genes are most significantly enriched is considered the “synapse module”, while the “HDAC2&Sp3 module” contains both HDAC2 and Sp3. Synaptic genes were defined by SynSysNet. Expression of the eigengene representing the synapse module is anti-correlated with expression of the eigengene representing the HDAC2/Sp3 module (as highlighted with black dotted lines). The left black-white scale indicates the statistical=log₁₀P value for the enrichment of synaptic genes, which was generated by Fisher's exact test in R. The right black-white scale indicates the r value, the correlation coefficient between two eigengenes. FIG. 3E shows heat maps of expression levels of genes in HDAC2&Sp3 module (left) and synapse module (right). The thirteen columns to the left of each heat map are from control cases; the ten columns to the right are from AD patients.

FIGS. 4A-4D show elevated levels of Sp3 and HDAC2 impair synaptic plasticity in CK-p25 mice. FIG. 4A shows representative western blot images and quantification of Sp3 from the cortex of control and CK-p25 mice (n=3). The quantifications were done after normalizing to β-tubulin. * P<0.05 (two-tailed Student's t-test). FIG. 4B shows representative immunoblots and quantifications of Sp3 co-IPed with HDAC2 from cortical tissues from control and CK-p25 mice (n=6). IP was performed with anti-HDAC2 antibody (ab12169) or mouse IgG (Negative control). * P<0.05 (one-tailed Student's t-test). Values are means±s.e.m. FIG. 4C shows ChIP-qPCR results for HDAC2 (top panel) and Sp3 (bottom panel) at the promoters of their target genes and control genes in neurons sorted from cortex of control and CK-p25 mice (n=3). * P<0.05, ** P<0.01 (Dunnett's test). FIG. 4D shows field excitatory postsynaptic potential (fEPSP) slopes in hippocampal area CA1 of control and CK-p25 mice injected with control or Sp3 shRNAs. Slopes were normalized by the average of slopes before 2× theta-burst stimulation (TBS) (n=5-9 slices). * P<0.05 (Repeated measurement two-way ANOVA). Values are means±s.e.m.

FIGS. 5A-5E show the C-terminal region of HDAC2 is critical for regulation of synaptic function. FIG. 5A shows a diagram of the various HDAC2 and 1 chimera constructs. The regions labelled with # are identical between HDAC1 and 2. The regions filled with grey are from HDAC2, and the ones shaded with grey lines are from HDAC1. Two-way arrows indicate amplicons with qPCR primer sets used in FIGS. 5B-5C for HDAC1 and HDAC2, respectively. FIG. 5B shows quantitative RT-qPCR using primers detecting HDAC1 from primary neurons transduced with the indicated constructs. Values are means±s.e.m. FIG. 5C quantitative RT-qPCR using primers detecting HDAC2 from primary neurons transduced with the indicated constructs. Values are means±s.e.m. FIG. 5D shows representative mEPSC traces corresponding to the conditions shown in FIG. 5E. FIG. 5E shows the amplitude of mEPSCs following rescue of HDAC2-knockdown neurons with the indicated constructs (n=5-12). Solid and striped columns indicate no rescue and significant rescue, respectively. ** P<0.01 (Dunnett's test).

FIGS. 6A-6E show exogenous expression of HDAC2 C-terminal domain ameliorates synaptic and cognitive dysfunction in CK-p25 mice. FIG. 6A shows representative western blot images of co-immunoprecipitation of Sp3 or Sin3A with HDAC2, flag-tagged mCherry, 1C and 2C in Neuro2A cells. Arrows indicate the bands of mCherry-1C, mCherry-2C and mCherry, respectively. FIG. 6B shows representative traces and quantifications of the amplitude and frequency of mEPSCs from primary neurons transduced with control (mCherry) or 2C expressing virus (n=5-8). * P<0.05, ** P<0.01 (two-tailed Welch's t-test). FIG. 6C (top panel) shows ChIP-qPCR results of HDAC2 at the promoters of target genes and control genes in primary neurons transduced with control (mCherry) or 2C expressing virus (n=3). * P<0.05, ** P<0.01 (one-tailed Student's t-test). FIG. 6C (bottom panel) shows quantitative RT-qPCR results of the target genes and control genes in primary neurons transduced with 2C (n=4). Values are means±s.e.m. *P<0.05 (unpaired t-test corrected by Holm-Šídák method). FIG. 6D shows fEPSP slopes from hippocampal area CA1 of CK-p25 mice injected with control or 2C expressing lentivirus. Slopes were normalized to baseline for each slice before 2×TBS (n=5-6 slices). ** P<0.01 (Repeated measurement two-way ANOVA). FIG. 6E shows freezing responses of CK (control mice) and CK-p25 mice injected with control or 2C expressing virus, 24h after contextual fear conditioning (n=10 CK-p25 mice each, n=8 CK mice). * P<0.05 (Turkey's test). Values are means±s.e.m.

FIGS. 7A-7D show the scheme of screening for HDAC2-interacting partners using weighted gene co-expression network analysis. FIG. 7A shows unbiased clustering of high- and low-HDAC2 expressing individuals based on global gene expression patterns reliably separates the two groups. Dark grey and light grey indicate individuals with high and low HDAC2 expression, respectively. FIG. 7B shows the gene dendrogram and co-expression modules. Each color indicates a distinct module containing genes with highly correlated expression (the HDAC2-containing module is indicated in grey. FIG. 7C shows a heat map of pearson's correlation coefficients between expression of the “repressors” (x-axis) and all genes (y-axis) in the HDAC2 module. Classifications were based on gene ontology analysis for “repressors”. FIG. 7D shows representative western blot images of co-immunoprecipitations from mouse cortex using an HDAC2 antibody, performed to test the binding between HDAC2 and TDP2, a protein previously reported to interact with HDAC2.

FIGS. 8A-8D show knockdown efficiency and mEPSC recordings following knockdown of HDAC2 and candidate co-repressors. FIG. 8A shows the knockdown efficiencies of Hdac2 and Sp3 shRNAs (n=4). FIG. 8B shows the knockdown efficiencies of Sap30 and Ttrap shRNAs (n=2). FIG. 8C shows representative traces, mEPSC amplitude and frequency from neurons transduced with Sap30 or Ttrap (TDP2) shRNAs (n=6-10). n.s. means not significant (two-tailed Student's t-test). FIG. 8D shows the expression levels of Sp3 in neurons transduced with control shRNA, Sp3 shRNA or shRNA-resistant Sp3 combined with Sp3 shRNA (n=3). Values are means±s.e.m.

FIGS. 9A-9H show RNA-seq analysis of neurons treated with HDAC2 or Sp3 shRNAs. FIGS. 9A-9B are snapshots of RNA-seq trace files from neurons treated with control, HDAC2 or Sp3 shRNAs at HDAC2 showing reduction of the relevant transcripts. The data was from biological duplicates for each condition. FIGS. 9C-9D show immunoblots of HDAC2, Sp3 and actin from neurons transduced with the indicated shRNAs.

FIG. 9E shows a list of the “synaptic” genes selected for ChIP analysis. Expression of each gene was increased by both HDAC2 and Sp3 knockdown, as well as decreased in CK-p25 mice. The genes in bold were also decreased in AD patients. FIGS. 9F-9G shows RT-qPCR results of the target genes in primary neurons transduced with Sp3 or HDAC2 shRNAs (n=3-7). * P<0.05, ** P<0.01 (one-tailed Student's or Welch's t-test). Values are means±s.e.m. FIG. 9H shows a matrix that is a comparison of differentially expressed genes in the CK-p25 mouse with genes co-regulated by HDAC2 and Sp3. P-value is calculated by Fisher's exact test. Genes in black indicate no change in expression, dark grey indicates a decrease in expression, and light grey indicates an increase in expression.

FIGS. 10A-C show the correlation of ChIP signals of Sp3 and HDAC2 between hippocampus and cortex. FIG. 10A shows FACS plots for isolation of NeuN+ nuclei. FIGS. 10B-10C shows ChIP-qPCR results of HDAC2 (FIG. 10B) and Sp3 (FIG. 10C) at the promoters or downstream regions of their target genes, and control genes, in neurons sorted from mouse hippocampus (n=3). Values are means±s.e.m. FIG. 10C shows the correlation of ChIP signals between hippocampus and cortex for HDAC2 (left panel), Sp3 (middle panel) and IgG (right panel).

FIGS. 11A-11D show elevated levels of HDAC2 and Sp3 in CK-p25 mice. FIGS. 11A-11B shows representative immunoblots and quantifications of HDAC2 in the cortex (FIG. 11A) as well as HDAC2 and Sp3 levels in the hippocampus (FIG. 11B) of control (CK) and CK-p25 mice (n=3). The quantifications were done after normalizing to (3-tubulin. * P<0.05, ** P<0.01 (two-tailed Student's t-test). FIG. 11C shows representative immunoblots and quantifications of Sp3 co-IPed with HDAC2 from hippocampal tissue from control and CK-p25 mice (n=3). IP was performed with anti-HDAC2 antibody (ab12169) or mouse IgG (Negative control). ** P<0.01 (one-tailed Student's t-test). Values are means±s.e.m. FIG. 11D shows FACS plots for isolation of NeuN+ nuclei from CK and CK-p25 mice.

FIGS. 12A-12C shows knockdown of Sp3 in vivo. FIG. 12A shows representative immunohistochemical images of Sp3 and copGFP (transduction marker induced by an independent promoter in the same vector as the shRNA) in hippocampal CA1 of mice injected with control shRNA and Sp3 shRNA. FIG. 12B shows a western blot of HDAC2, Sp3 and internal controls in copGFP-positive regions of hippocampal CA1. FIG. 12C shows input-output curves following stimulation of the Schaffer collateral pathway in hippocampal slices from control (CK) and CK-p25 mice injected with control or Sp3 shRNA. Values are means±s.e.m.

FIGS. 13A-13C show the effects of exogenous expression of HDAC2 C-terminal fragment (2C). FIG. 13A shows proliferation ratios of MEFs transduced with control shRNA, HDAC2 shRNA, HDAC2+HDAC1 shRNA, mCherry (control for 2C) or 2C. ** P<0.01 (Dunnett's test), n.s.; not significant (one-tailed Student's t-test). FIG. 13B shows input-output curves following stimulation of the Schaffer collateral pathway in hippocampal slices from CK-p25 transduced with control or 2C. FIG. 13C shows freezing responses to the auditory cue by control mice and CK-p25 mice transduced with control or 2C, measured 48h after cued fear conditioning (n=8 or 10). * P<0.05 (Turkey's test). Values are means±s.e.m.

### DETAILED DESCRIPTION

Epigenetic mechanisms such as histone acetylation are critical modulators of transcriptional activity regulating diverse biological processes. Among histone-modifying enzymes, HDAC2 is a critical negative regulator of structural and functional plasticity in the mammalian nervous system. HDAC2 localizes to the promoters of numerous synaptic plasticity associated genes where it promotes localized deacetylation of histone substrates (Graff et al., 2012, Nature 483,p. 222-226). Consistently, loss of HDAC2 or HDAC inhibitor treatments promote synaptic gene expression, long term synaptic plasticity and memory processes, while HDAC2 overexpression has opposing effects (Fischer et al., 2007, Nature 447, p. 178-182; Graff et al., 2014 Cell 156, p. 261-276; Graff et al., 2012, Nature 483, p. 222-226; Guan et al., Nature, 2009).

A major hurdle to the treatment of neurodegenerative disease by targeting HDAC2 however, is the lack of specificity of current HDAC inhibitor compounds. These compounds target the deacetylase catalytic domain, and a number of them exhibit selectivity for the class I HDACs (HDACs 1, 2, 3 and 8) over class II, III and IV enzymes, but functional HDAC2 specific inhibitors have yet to be reported. This lack of specificity is particularly problematic given the distinct and sometimes opposing functions of the different HDAC enzymes (Dobbin et al., 2013 Nature Neuroscience, 16, p. 1008-1015; Wang et al., 2013, Cell, 138 p. 1019-1031). Further complicating matters is the large number of different chromatin binding complexes HDAC enzymes can participate in. Indeed, HDAC2 and other HDACs often interact with different binding partners and regulate distinct subsets of genes depending on cell-type, developmental stage, and any number of other intrinsic or extrinsic signals.

A class of HDAC2 inhibitors which are both capable of inhibiting HDAC2 complexes to enhance cognitive function and avoiding the adverse side effects of available pan-HDAC inhibitors have been discovered according to the invention. This group of compounds are able to specifically disrupt the interaction of HDAC2 with the DNA binding proteins(s) responsible for recruitment of HDAC2 to the promoters of synaptic plasticity-associated genes. It was demonstrated herein that knockdown of the transcription factor Sp3 was similar to HDAC2 knockdown in its ability to facilitate synaptic transmission. Consistent with a role in recruitment of HDAC2 to target genes, knockdown of Sp3 was able to reduce HDAC2 occupancy and increase histone acetylation at synaptic gene promoters, as well as antagonizing synaptic gene expression. Also like HDAC2, it was found that Sp3 expression was elevated in the brain of a mouse model of AD-like neurodegeneration, as well as in patients having Alzheimer's disease. Importantly, exogenous expression of an HDAC2 inhibitor of the invention which disrupts HDAC2-Sp3 interaction was able to counteract the synaptic plasticity and memory defects found in a mouse model of Alzheimer's-like neurodegeneration.

Thus, in some aspects, the invention is methods and compositions for disrupting HDAC2-Sp3 interactions. HDAC2 is a histone deacetylase that is recruited to the promoters of synaptic plasticity genes by the transcription factor Sp3. The term “HDAC2” used herein encompasses HDAC2 from various species, for example, human HDAC2. As an example, the amino acid sequence of human HDAC2 is provided in GenBank accession number NP_001518.3 and UniProtKB number Q92769.

HDAC2-specific inhibition is problematic due to the high conservation of active sites among mammalian HDAC isoforms. Accordingly, current HDAC inhibitors lack specificity toward HDAC2 and inhibit multiple HDACs, which can be deleterious considering the diverse functions of HDAC enzymes throughout the body. For example, in the context of neuronal function, loss of HDAC2 promotes synaptic gene expression and memory processes, but during hematopoiesis, loss of HDAC1 and HDAC2 leads to defects in differentiation and thrombocytopenia. Currently available pan-HDAC inhibitors interrupt cell proliferation, and consequently have been used as anti-cancer agents.

As described herein, specific proteins within the HDAC2 complex that control synaptic gene expression were identified, thereby providing targets for relieving HDAC2 mediated repression of neuronal genes during neurodegeneration while maintaining HDAC2 functions in other processes.

Accordingly, the present disclosure provides methods of treating a neurodegenerative disease (e.g., alleviating neurodegeneration, delaying the onset of degeneration, and/or suppressing degeneration) in a subject using an effective amount of inhibitory compounds, including HDAC2/Sp3 inhibitors which can inhibit HDAC2 interaction with Sp3, HDAC2 localization inhibitors, which can reduce or inhibit the localization of HDAC2 to chromatin, or Sp3 expression inhibitors, which reduce levels of Sp3 available for HDAC2 binding.

### HDAC2 Inhibitors and Pharmaceutical Compositions

The compounds useful according to the invention are specific inhibitors of HDAC2 activity. A specific inhibitor of HDAC2 activity is a compound that interrupts or interferes with HDAC2 activity without influencing cellular proliferation or HDAC1 activity. Specific inhibitors of HDAC2 activity include but are not limited to HDAC2/Sp3 inhibitors, HDAC2 localization inhibitors and Sp3 expression inhibitors.

An HDAC2/Sp3 inhibitor as used herein refers to a compound that blocks, suppresses, or reduces binding interaction between HDAC2 and Sp3. The HDAC2/Sp3 inhibitor may reduce or interfere with HDAC2-Sp3 interactions through any mechanism including, but not limited to, binding to HDAC2 preventing HDAC2 from interacting with Sp3 and/or binding to Sp3 and preventing Sp3 binding to HDAC2

An HDAC2 localization inhibitor as used herein refers to a compound that blocks, suppresses, or reduces recruitment of HDAC2 to chromatin, thus interfering with HDAC2 recruitment to the promoters of synaptic plasticity genes. HDAC2 localization inhibitors include but are not limited to compounds that block, suppress, or reduce binding interaction between HDAC2 and chromatin recruitment factors, such as Sp3. In some embodiments the HDAC2 localization inhibitors include HDAC2/Sp3 inhibitors.

The terms reduce, interfere, inhibit, and suppress refer to a partial or complete decrease in activity levels relative to an activity level typical of the absence of the inhibitor. For instance, the decrease may be by at least 20%, 50%, 70%, 85%, 90%, 100%, 150%, 200%, 300%,or 500%, or by 10-fold, 20-fold, 50-fold, 100-fold, 1000-fold, or 10⁴-fold.

In some instances, a HDAC2/Sp3 inhibitor described herein may be an agent that binds to HDAC2 and inhibits binding of HDAC2 to Sp3. In other instances, a HDAC2/Sp3 inhibitor may be an agent that binds to Sp3 and interferes with the interaction between HDAC2 and Sp3. In other examples, a HDAC2 inhibitor may be an agent that inhibits HDAC2 interaction with Sp3 or expression of HDAC2 but does not significantly inhibit other HDAC enzymes from interaction with Sp3 or expression of any other HDAC enzymes such as HDAC1, HDAC3, HDAC4, HDAC5, HDAC6, HDAC7, HDAC8, HDAC9, HDAC10, HDAC11, HDAC12, HDAC13, HDAC14, HDAC15, HDAC16, HDAC17, or HDAC18.

Exemplary HDAC2/Sp3 inhibitors and HDAC2 localization inhibitors include, but are not limited to, peptides such as antibodies small molecule compounds, and other compounds which may disrupt HDAC2/SP3 interactions.

In some embodiments, the HDAC2/Sp3 inhibitor and/or HDAC2 localization inhibitor can be a peptide inhibitor that binds to HDAC2 or its binding partner, e.g., SP3 and disrupts the interaction between them. In particular it is demonstrated herein that the C-terminal portion of HDAC2 is responsible for the binding interaction with Sp3. The inhibitor which is a peptide may be a peptide which is a portion of the HDAC2 molecule involved in Sp3 binding, a portion of the Sp3 molecule involved in HDAC2 binding or any other peptide which may bind to those regions of HDAC2 or Sp3 and competitively inhibit or block the natural binding interaction, such as an antibody or fragment thereof or may bind to another factor that will disrupt the binding between HDAC2 and Sp3.

Thus, in some embodiments the peptide comprises a portion of the HDAC2 protein, wherein the peptide specifically binds to Sp3 and blocks its interaction with full-length HDAC2 protein. In some embodiments, provided herein are peptide inhibitors comprising the C-terminal fragment of HDAC2. The peptide inhibitors referred to herein can be from any source. In some embodiments, the peptide inhibitors are from primates or rodents. In some embodiments, the peptide inhibitors are from mouse or rat. In some embodiments, the peptide inhibitors are from human.

In some embodiments, the peptide inhibitor comprises the C-terminal fragment of HDAC2 having an amino acid that is at least 80% identical to SEQ ID NO: 1. Amino acids 1 to 98 in SEQ ID NO: 1 correspond to positions 390-488 of the human HDAC2 sequence.

In some embodiments, the peptide comprises a sequence that has at least about 50%, 55%, 60%, 65%, 70%, 75%, 80%, 85%, 90%, 95%, or 99% sequence identity to the amino acid sequence of SEQ ID NO: 1. In some embodiments, the peptide comprises a sequence that has about 50% to about 99%, about 60% to about 99%, about 70% to about 99%, about 75% to about 99%, about 80% to about 99%, about 85% to about 99%, about 90% to about 99%, about 95% to about 99% sequence identity to the amino acid sequence of SEQ ID NO: 1. In some embodiments, the peptide has one or more amino acid substitutions from SEQ ID NO: 1 or fragments thereof, such that the peptide is not a fragment of a naturally occurring peptide.

In some embodiments, the peptide is about 25-110 amino acids in length. In some embodiments, the peptide is about 35-110, about 45-110, about 55-110, about 65-110, about 75-110, about 85-110, about 95-110, or about 100-110 amino acids in length. In some embodiments, the peptide is about 25-100, about 25-90, about 25-80, about 25-70, about 25-60, about 25-50, about 25-40, or about 25-30 amino acids in length.

In some embodiments, the peptide comprises at least one unnatural amino acid. In some embodiments, the peptide comprises one or two unnatural amino acids. In some embodiments, the peptide comprises at least one D-amino acid. In some embodiments, the peptide comprises one or two D-amino acids. In some embodiments, the peptide comprises 1-5 D-amino acids. In some embodiments, the peptide comprises 1-10 D-amino acids. In some embodiments, the peptide comprises all D-amino acids. In some embodiments, the peptide comprises at least 2000 Da in molecular weight.

The peptides described herein can comprise L-amino acids, D-amino acids, or combinations thereof. In certain embodiments, all the residues in the peptide are L-amino acids. In certain embodiments, all the residues in the peptide are D-amino acids. In certain embodiments, the residues in the peptide are a combination of L-amino acids and D-amino acids. In certain embodiments, the peptides contain 1 to 5 residues that are D-amino acids. In certain embodiments, at least 5% of the peptide sequence comprises D-amino acids. In certain embodiments, at least 10% of the peptide sequence comprises D-amino acids. In certain embodiments, at least 20% of the peptide sequence comprises D-amino acids. In certain embodiments, at most 15% of the peptide sequence comprises D-amino acids. In certain embodiments, at most 20% of the peptide sequence comprises D-amino acids. In certain embodiments, at most 50% of the peptide sequence comprises D-amino acids. In certain embodiments, at most 60% of the peptide sequence comprises D-amino acids. In certain embodiments, at most 80% of the peptide sequence comprises D-amino acids. In certain embodiments, at most 90% of the peptide sequence comprises D-amino acids. In certain embodiments, about 5-15% of the peptide sequence comprises D-amino acids. In certain embodiments, about 5-20% of the peptide sequence comprises D-amino acids. In certain embodiments, about 5-50% of the peptide sequence comprises D-amino acids.

In some embodiments, the peptide comprises the amino acid sequence of SEQ ID NO: 1 with 1, 5, 10, 15, 20, or 25 amino acid changes (e.g., amino acid substitutions, deletions, and/or additions). In some embodiments, the amino acid change is an amino acid substitution in which 1, 5, 10, 15, 20, or 25 amino acids are mutated to another amino acid. In some embodiments, the amino acid change is an addition or deletion, where the addition or deletion comprises adding or deleting up to 1, 5, 10, 15, 20, or 25 residues at the point of mutation in the wild type sequence. The residues being added or deleted can be consecutive or non-consecutive residues.

In certain embodiments, the peptide has a solubility of up to about 30 mg/mL, about 40 mg/mL, about 50 mg/mL, about 60 mg/mL, about 100 mg/mL, or about 120 mg/mL in aqueous solution.

In certain embodiments, the peptide exhibits at least 30%, 40%, 50%, 60%, 70%, 80%, 85%, 90%, or 95% inhibition of HDAC2 binding to Sp3. In certain embodiments, the peptide exhibits at least 70% inhibition of HDAC2 binding to Sp3. In certain embodiments, the peptide exhibits at least 80% inhibition of HDAC2 binding to Sp3. Various methods are known for measuring the inhibitory activity. For example, inhibitor activity can be measured with chromatin immunoprecipitation experiments using cultured cells expressing the peptide inhibitor, e.g., Example 5 described herein. A reduction of HDAC2 enrichment at the promoters of genes indicates inhibitor activity.

HDAC2/Sp3 inhibitors include antibodies and fragments thereof, such as anti-HDAC2 and/or anti-Sp3 antibodies may be used in the methods described herein. In some embodiments the anti-HDAC2 antibody specifically binds to HDAC2 and prevents the interaction between HDAC2 and Sp3. In some embodiments the anti-Sp3 antibody specifically binds to Sp3 and prevents the interaction between HDAC2 and Sp3. In other embodiments the antibody is a bifunctional antibody capable of binding both HDAC2 and Sp3.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule.

As used herein, the term “antibody” encompasses not only intact (i.e., full-length) polyclonal or monoclonal antibodies, but also antigen-binding fragments thereof (such as Fab, Fab′, F(ab′)₂, Fv), single chain (scFv), mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

An anti-HDAC2 antibody is an antibody capable of binding to HDAC2, which may reduce HDAC2 binding to Sp3 and/or inhibit HDAC2 biological activity. In some examples, an anti-HDAC2 antibody used in the methods described herein reduces HDAC2 binding to Sp3 by at least 20%, at least 40%, at least 50%, at least 75%, at least 90%, at least 100%, or by at least 2-fold, at least 5-fold, at least 10-fold, at least 20-fold, at least 50-fold, at least 100-fold, or at least 1000-fold.

An anti-Sp3 antibody is an antibody capable of binding to Sp3, which may reduce HDAC2 binding to Sp3 and/or inhibit Sp3 biological activity. In some examples, an anti-Sp3 antibody used in the methods described herein reduces HDAC2 binding to Sp3 by at least 20%, at least 40%, at least 50%, at least 75%, at least 90%, at least 100%, or by at least 2-fold, at least 5-fold, at least 10-fold, at least 20-fold, at least 50-fold, at least 100-fold, or at least 1000-fold.

The binding affinity of an anti-HDAC2 or Sp3 antibody to HDAC2 or Sp3 (such as human HDAC2 or Sp3) can be less than any of about 100 nM, about 50 nM, about 10 nM, about 1 nM, about 500 pM, about 100 pM, or about 50 pM to any of about 2 pM. Binding affinity can be expressed K_{D} or dissociation constant, and an increased binding affinity corresponds to a decreased K_{D}. One way of determining binding affinity of antibodies to HDAC2 or Sp3 is by measuring binding affinity of monofunctional Fab fragments of the antibody. To obtain monofunctional Fab fragments, an antibody (for example, IgG) can be cleaved with papain or expressed recombinantly. The affinity of an anti-HDAC2 or Sp3Fab fragment of an antibody can be determined by surface plasmon resonance (BIAcore3000™ surface plasmon resonance (SPR) system, BIAcore, INC, Piscaway N.J.). Kinetic association rates (kₒₙ) and dissociation rates (k_{off}) (generally measured at 25° C.) are obtained; and equilibrium dissociation constant (K_{D}) values are calculated as k_{off}/kₒₙ.

In some embodiments, the antibody binds human HDAC2 or Sp3, and does not significantly bind a HDAC2 or Sp3 from another mammalian species. In some embodiments, the antibody binds human HDAC2 or Sp3 as well as one or more HDAC2 or Sp3 from another mammalian species. In still other embodiments, the antibody binds HDAC2 and does not significantly cross-react with other proteins such as other HDACs. The epitope(s) bound by the antibody can be continuous or discontinuous.

The anti-HDAC2 or Sp3 antibodies to be used in the methods described herein can be murine, rat, human, or any other origin (including chimeric or humanized antibodies). In some examples, the antibody comprises a modified constant region, such as a constant region that is immunologically inert, e.g., does not trigger complement mediated lysis, or does not stimulate antibody-dependent cell mediated cytotoxicity (ADCC). ADCC activity can be assessed using methods disclosed in U.S. Pat. No. 5,500,362. In other embodiments, the constant region is modified as described in Eur. J. Immunol. (1999) 29:2613-2624; PCT Application No. PCT/GB99/01441; and/or UK Patent Application No. 9809951.8.

Any of the antibodies described herein can be either monoclonal or polyclonal. A “monoclonal antibody” refers to a homogenous antibody population and a “polyclonal antibody” refers to a heterogenous antibody population. These two terms do not limit the source of an antibody or the manner in which it is made.

In some embodiments, the antibody used in the methods described herein is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs “derived from” one or more CDRs from the original antibody. Humanized antibodies may also involve affinity maturation.

In some embodiments, the antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some examples, the antibody disclosed herein specifically binds a target antigen, such as human HDAC2 or Sp3. An antibody that “specifically binds” (used interchangeably herein) to a target or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target antigen if it binds with greater affinity, avidity, more readily, and/or with greater duration than it binds to other substances. For example, an antibody that specifically (or preferentially) binds to a HDAC2 or Sp3 epitope is an antibody that binds this HDAC2 or Sp3 epitope with greater affinity, avidity, more readily, and/or with greater duration than it binds to other HDAC2 or Sp3epitopes or non-HDAC2 or Sp3 epitopes. It is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding.

Antibodies capable of reducing HDAC2 binding to Sp3 can be an antibody that binds a HDAC2 or Sp3 (e.g., a human HDAC2 or Sp3) and inhibits HDAC2 biological activity and/or Sp3 mediated recruitment of HDAC2 to promotors of genes. Antibodies capable of reducing binding of HDAC2 to Sp3 (e.g., anti-HDAC2 or Sp3 antibodies) as described herein can be made by any method known in the art.

The ability of an antibody or fragment thereof to bind to HDDAC2 or Sp3 and function according to the methods of the invention can be assayed using known binding or activity assays, such as those described herein. Alternatively, competition assays can be performed using other antibodies known to bind to the same antigen to determine whether an antibody binds to the same epitope as the other antibodies. Competition assays are well known to those of skill in the art.

HDAC2/Sp3 inhibitors also include small molecule inhibitors that directly inhibit HDAC2 binding to Sp3, or other agents that inhibit the binding interaction.

The HDAC2/Sp3 inhibitory compounds of the invention may exhibit any one or more of the following characteristics: (a) reduces HDAC2 binding to Sp3; (b) prevents, ameliorates, or treats any aspect of a neurodegenerative disease; (c) reduces synaptic dysfunction; (d) reduces cognitive dysfunction; (e) reduces histone deacetylation; (f) reduces recruitment of HDAC2 to promoters of genes. One skilled in the art can prepare such inhibitory compounds using the guidance provided herein.

In other embodiments, the HDAC2 inhibitory compounds described herein are small molecules, which can have a molecular weight of about any of 100 to 20,000 daltons, 500 to 15,000 daltons, or 1000 to 10,000 daltons. Libraries of small molecules are commercially available. The small molecules can be administered using any means known in the art, including inhalation, intraperitoneally, intravenously, intramuscularly, subcutaneously, intrathecally, intraventricularly, orally, enterally, parenterally, intranasally, or dermally. In general, when the HDAC2 inhibitor according to the invention is a small molecule, it will be administered at the rate of 0.1 to 300 mg/kg of the weight of the patient divided into one to three or more doses. For an adult patient of normal weight, doses ranging from 1 mg to 5 g per dose can be administered.

The above-mentioned small molecules can be obtained from compound libraries. The libraries can be spatially addressable parallel solid phase or solution phase libraries. See, e.g., Zuckermann et al. J. Med. Chem. 37, 2678-2685, 1994; and Lam Anticancer Drug Des. 12:145, 1997. Methods for the synthesis of compound libraries are well known in the art, e.g., DeWitt et al. PNAS USA 90:6909, 1993; Erb et al. PNAS USA 91:11422, 1994; Zuckermann et al. J. Med. Chem. 37:2678, 1994; Cho et al. Science 261:1303, 1993; Carrell et al. Angew Chem. Int. Ed. Engl. 33:2059, 1994; Carell et al. Angew Chem. Int. Ed. Engl. 33:2061, 1994; and Gallop et al. J. Med. Chem. 37:1233, 1994. Libraries of compounds may be presented in solution (e.g., Houghten Biotechniques 13:412-421, 1992), or on beads (Lam Nature 354:82-84, 1991), chips (Fodor Nature 364:555-556, 1993), bacteria (U.S. Pat. No. 5,223,409), spores (U.S. Pat. No. 5,223,409), plasmids (Cull et al. PNAS USA 89:1865-1869, 1992), or phages (Scott and Smith Science 249:386-390, 1990; Devlin Science 249:404-406, 1990; Cwirla et al. PNAS USA 87:6378-6382, 1990; Felici J. Mol. Biol. 222:301-310, 1991; and U.S. Pat. No. 5,223,409).

Alternatively, the inhibitors described herein may be Sp3 expression inhibitors that decreases Sp3 expression, for example, morpholino oligonucleotides, small interfering RNA (siRNA or RNAi), antisense nucleic acids, or ribozymes. RNA interference (RNAi) is a process in which a dsRNA directs homologous sequence-specific degradation of messenger RNA. In mammalian cells, RNAi can be triggered by 21-nucleotide duplexes of small interfering RNA (siRNA) without activating the host interferon response. The dsRNA used in the methods disclosed herein can be a siRNA (containing two separate and complementary RNA chains) or a short hairpin RNA (i.e., a RNA chain forming a tight hairpin structure), both of which can be designed based on the sequence of the target gene.

Optionally, a nucleic acid molecule to be used in the method described herein (e.g., an antisense nucleic acid, a small interfering RNA, or a microRNA) as described above contains non-naturally-occurring nucleobases, sugars, or covalent internucleoside linkages (backbones). Such a modified oligonucleotide confers desirable properties such as enhanced cellular uptake, improved affinity to the target nucleic acid, and increased in vivo stability.

In one example, the nucleic acid has a modified backbone, including those that retain a phosphorus atom (see, e.g., U.S. Pat. Nos. 3,687,808; 4,469,863; 5,321,131; 5,399,676; and 5,625,050) and those that do not have a phosphorus atom (see, e.g., U.S. Pat. Nos. 5,034,506; 5,166,315; and 5,792,608). Examples of phosphorus-containing modified backbones include, but are not limited to, phosphorothioates, chiral phosphorothioates, phosphorodithioates, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having 3′-5′ linkages, or 2′-5′ linkages. Such backbones also include those having inverted polarity, i.e., 3′ to 3′, 5′ to 5′ or 2′ to 2′ linkage. Modified backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In another example, the nucleic acid used in the disclosed methods includes one or more substituted sugar moieties. Such substituted sugar moieties can include one of the following groups at their 2′ position: OH; F; O-alkyl, S-alkyl, N-alkyl, O-alkenyl, S-alkenyl, N-alkenyl; O-alkynyl, S-alkynyl, N-alkynyl, and O-alkyl-O-alkyl. In these groups, the alkyl, alkenyl and alkynyl can be substituted or unsubstituted C₁ to C₁₀ alkyl or C₂ to C₁₀ alkenyl and alkynyl. They may also include at their 2′ position heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide. Preferred substituted sugar moieties include those having 2′-methoxyethoxy, 2′-dimethylaminooxyethoxy, and 2′-dimethylaminoethoxyethoxy. See Martin et al., Helv. Chim. Acta, 1995, 78, 486-504.

In yet another example, the nucleic acid includes one or more modified native nucleobases (i.e., adenine, guanine, thymine, cytosine and uracil). Modified nucleobases include those described in U.S. Pat. No. 3,687,808, The Concise Encyclopedia Of Polymer Science And Engineering, pages 858-859, Kroschwitz, J. I., ed. John Wiley & Sons, 1990, Englisch et al., Angewandte Chemie, International Edition, 1991, 30, 613, and Sanghvi, Y. S., Chapter 15, Antisense Research and Applications, pages 289-302, CRC Press, 1993. Certain of these nucleobases are particularly useful for increasing the binding affinity of the antisense oligonucleotide to its target nucleic acid. These include 5-substituted pyrimidines, 6-azapyrimidines and N-2, N-6 and O-6 substituted purines (e.g., 2-aminopropyl-adenine, 5-propynyluracil and 5-propynylcytosine). See Sanghvi, et al., eds., Antisense Research and Applications, CRC Press, Boca Raton, 1993, pp. 276-278).

Any of the nucleic acids can be synthesized by methods known in the art. See, e.g., Caruthers et al., 1992, Methods in Enzymology 211, 3-19, Wincott et al., 1995, Nucleic Acids Res. 23, 2677-2684, Wincott et al., 1997, Methods Mol. Bio. 74, 59, Brennan et al., 1998, Biotechnol Bioeng., 61, 33-45, and Brennan, U.S. Pat. No. 6,001,311. It can also be transcribed from an expression vector and isolated using standard techniques.

The inhibitors described herein can be identified or characterized using methods known in the art, whereby reduction, amelioration, or neutralization of HDAC2 binding to Sp3 is detected and/or measured. For example, an ELISA-type assay may be suitable for qualitative or quantitative measurement of HDAC2 binding to Sp3.

The HDAC2/Sp3 inhibitors can also be identified by incubating a candidate agent with HDAC2 and monitoring any one or more of the following characteristics: (a) binds to HDAC2; (b) reduces HDAC2 binding to Sp3; (c) prevents, ameliorates, or treats any aspect of a neurodegenerative disease; (d) preserves cognitive function; (e) preserves histone acetylation; (f) reduces recruitment of HDAC2 to promoters of genes; (g) inhibits (reduces) HDAC2 synthesis, production or release.

In some embodiments, a HDAC2/Sp3 inhibitor is identified by incubating a candidate agent with HDAC2 and monitoring binding and attendant reduction or neutralization of binding to Sp3. The binding assay may be performed with purified HDAC2 polypeptide(s), or with cells naturally expressing, or transfected to express, HDAC2 polypeptide(s). In one embodiment, the binding assay is a competitive binding assay, where the ability of a candidate antibody to compete with a known HDAC2 inhibitor for HDAC2 binding is evaluated. The assay may be performed in various formats, including the ELISA format. In other embodiments, a HDAC2 inhibitor is identified by incubating a candidate agent with HDAC2 and monitoring attendant inhibition of HDAC2/Sp3 complex formation. Following initial identification, the activity of a candidate HDAC2 inhibitor can be further confirmed and refined by bioassays, known to test the targeted biological activities. Alternatively, bioassays can be used to screen candidates directly.

The examples provided below provide a number of assays that can be used to screen candidate HDAC2/Sp3 inhibitors. Bioassays include but are not limited to assaying, in the presence of a HDAC2 inhibitor, preservation of cognitive function and/or histone acetylation at gene promoters. In addition, Real-Time PCR (RT-PCR) can be used to directly measure Sp3 expression.

Further, a suitable HDAC2 inhibitor may be screened from a combinatory compound library using any of the assay methods known in the art and/or described herein.

### Pharmaceutical Compositions

One or more of the HDAC2 inhibitors described herein can be mixed with a pharmaceutically acceptable carrier (excipient), including buffer, to form a pharmaceutical composition for use in reducing HDAC2 binding to Sp3. “Acceptable” means that the carrier must be compatible with the active ingredient of the composition (and preferably, capable of stabilizing the active ingredient) and not deleterious to the subject to be treated. As used herein a pharmaceutically acceptable carrier does not include water and is more than a naturally occurring carrier such as water. In some embodiments the pharmaceutically acceptable carrier is a formulated buffer, a nanocarrier, an IV solution etc.

Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover. For example, a pharmaceutical composition described herein contains one or more HDAC2/Sp3 inhibitors such as peptide inhibitors that recognize different epitopes of the target antigen.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20^{th} Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ (polysorbate), PLURONICS™ (poloxamers) or polyethylene glycol (PEG). Pharmaceutically acceptable excipients are further described herein.

In some examples, the pharmaceutical composition described herein comprises liposomes containing the HDAC2 Sp3 inhibitor, which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active ingredients (e.g., an HDAC2 inhibitor) may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20^{th} Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., TWEEN™ 20, 40, 60, 80 or 85) and other sorbitans (e.g., SPAN™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as INTRALIPID™, LIPOSYN™, INFONUTROL™, LIPOFUNDIN™ and LIPIPHYSAN™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 .im, particularly 0.1 and 0.5 .im, and have a pH in the range of 5.5 to 8.0.

The emulsion compositions can be those prepared by mixing a HDAC2 inhibitor with Intralipid™ (a lipid emulsion) or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulised by use of gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

### Use of HDAC2 Inhibitors for Treating Neurodegenerative Disease

To practice the method disclosed herein, an effective amount of the pharmaceutical composition described above can be administered to a subject (e.g., a human) in need of the treatment via a suitable route (e.g., intravenous administration).

The subject to be treated by the methods described herein can be a human patient having, suspected of having, or at risk for a neurodegenerative disease. Examples of a neurodegenerative disease include, but are not limited to, MCI (mild cognitive impairment), post-traumatic stress disorder (PTSD), Alzheimer's Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, vascular dementia, progressive supranuclear palsy or cortical basal degeneration.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a neurodegenerative disease (e.g., MCI). A subject having a neurodegenerative disease can be identified by routine medical examination, e.g., clinical exam, medical history, laboratory tests, MRI scans, CT scans, or cognitive assessments. A subject suspected of having a neurodegenerative disease might show one or more symptoms of the disorder, e.g., memory loss, confusion, depression, short-term memory changes, and/or impairments in language, communication, focus and reasoning. A subject at risk for a neurodegenerative disease can be a subject having one or more of the risk factors for that disorder. For example, risk factors associated with neurodegenerative disease include (a) age, (b) family history, (c) genetics, (d) head injury, and (e) heart disease.

“An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a neurodegenerative disease. Alternatively, sustained continuous release formulations of an HDAC2 inhibitor may be appropriate. Various formulations and devices for achieving sustained release are known in the art.

In one example, dosages for a HDAC2 inhibitor as described herein may be determined empirically in individuals who have been given one or more administration(s) of HDAC2 inhibitor. Individuals are given incremental dosages of the inhibitor. To assess efficacy of the inhibitor, an indicator of a neurodegenerative disease (such as cognitive function) can be followed.

Generally, for administration of any of the peptide inhibitors described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a neurodegenerative disease, or a symptom thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the peptide inhibitor used) can vary over time.

When the HDAC2 inhibitor is not a peptide inhibitor, it may be administered at the rate of about 0.1 to 300 mg/kg of the weight of the patient divided into one to three doses, or as disclosed herein. In some embodiments, for an adult patient of normal weight, doses ranging from about 0.3 to 5.00 mg/kg may be administered. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of a HDAC2 inhibitor will depend on the specific HDAC2 inhibitor(s) (or compositions thereof) employed, the type and severity of neurodegenerative disease, whether the inhibitor is administered for preventive or therapeutic purposes, previous therapy, the patient's clinical history and response to the inhibitor, and the discretion of the attending physician. Typically the clinician will administer a HDAC2 inhibitor, such as a peptide inhibitor comprising the C-terminus of HDAC2, until a dosage is reached that achieves the desired result.

Administration of a HDAC2 inhibitor can be continuous or intermittent, depending, for example, upon the recipient's physiological condition, whether the purpose of the administration is therapeutic or prophylactic, and other factors known to skilled practitioners. The administration of a HDAC2 inhibitor (for example if the HDAC2 inhibitor is a peptide inhibitor) may be essentially continuous over a preselected period of time or may be in a series of spaced dose, e.g., either before, during, or after developing neurodegenerative disease.

As used herein, the term “treating” refers to the application or administration of a composition including one or more active agents to a subject, who has a neurodegenerative disease, a symptom of a neurodegenerative disease, or a predisposition toward a neurodegenerative disease, with the purpose to cure, heal, alleviate, relieve, alter, remedy, ameliorate, improve, or affect the disorder, the symptom of the disease, or the predisposition toward a neurodegenerative disease.

Alleviating a neurodegenerative disease includes delaying the development or progression of the disease, or reducing disease severity. Alleviating the disease does not necessarily require curative results. As used therein, “delaying” the development of a disease (such as MCI) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a neurodegenerative disease includes initial onset and/or recurrence.

In some embodiments, the HDAC2 inhibitor (e.g., a HDAC2 peptide inhibitor) described herein is administered to a subject in need of the treatment at an amount sufficient to reduce HDAC2 binding to Sp3 by at least 20% (e.g., 30%, 40%, 50%, 60%, 70%, 80%, 90% or greater). In other embodiments, the HDAC2 inhibitor is administered in an amount effective in preserving histone acetylation at gene promoters. Alternatively, the HDAC2 inhibitor is administered in an amount effective in reducing recruitment of HDAC2 to gene promoters.

In some embodiments, the HDAC2 inhibitor is administered to a subject in need of the treatment at an amount sufficient to enhance synaptic memory function by at least 20% (e.g., 30%, 40%, 50%, 60%, 70%, 80%, 90% or greater). Synaptic function refers to the ability of the synapse of a cell (e.g., a neuron) to pass an electrical or chemical signal to another cell (e.g., a neuron). Synaptic function can be determined by a conventional assay or by the assays described herein (see Examples).

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, a HDAC2 inhibitor is administered via site-specific or targeted local delivery techniques. Examples of site-specific or targeted local delivery techniques include various implantable depot sources of the HDAC2 inhibitor or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The therapeutic polynucleotides and polypeptides described herein can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent No. 0524968. Additional approaches are described in Philip, Mol. Cell. Biol. (1994) 14:2411, and in Woffendin, Proc. Natl. Acad. Sci. (1994) 91:1581.

It is also apparent that an expression vector can be used to direct expression of any of the protein-based HDAC2 inhibitors described herein (e.g., a peptide inhibitor). For example, other HDAC2 inhibitors that are capable of blocking (from partial to complete blocking) HDAC2 and/or a HDAC2 biological activity are known in the art.

The particular dosage regimen, i.e., dose, timing and repetition, used in the method described herein will depend on the particular subject and that subject's medical history.

In some embodiments, more than one HDAC2 inhibitor, such as an antibody and a small molecule HDAC2 inhibitory compound, may be administered to a subject in need of the treatment. The inhibitor can be the same type or different from each other. At least one, at least two, at least three, at least four, at least five different HDAC2 inhibitors can be co-administered. Generally, those HDAC2 inhibitors have complementary activities that do not adversely affect each other. HDAC2 inhibitors can also be used in conjunction with other agents that serve to enhance and/or complement the effectiveness of the agents.

Treatment efficacy can be assessed by methods well-known in the art, e.g., monitoring synaptic function or memory loss in a patient subjected to the treatment. See, e.g., Example 5.

### Combination Therapy

Also provided herein are combined therapies using any of the HDAC2 inhibitors described herein and another anti-neurodegenerative disease therapeutic agent, such as those described herein. The term combination therapy, as used herein, embraces administration of these agents (e.g., a HDAC2 inhibitor and an anti-neurodegenerative disease therapeutic agent) in a sequential manner, that is, wherein each therapeutic agent is administered at a different time, as well as administration of these therapeutic agents, or at least two of the agents, in a substantially simultaneous manner.

Sequential or substantially simultaneous administration of each agent can be affected by any appropriate route including, but not limited to, oral routes, intravenous routes, intramuscular, subcutaneous routes, and direct absorption through mucous membrane tissues. The agents can be administered by the same route or by different routes. For example, a first agent (e.g., a HDAC2 inhibitor) can be administered orally, and a second agent (e.g., an anti-neurodegenerative disease agent) can be administered intravenously.

As used herein, the term “sequential” means, unless otherwise specified, characterized by a regular sequence or order, e.g., if a dosage regimen includes the administration of a HDAC2 inhibitor and an anti-neurodegenerative disease agent, a sequential dosage regimen could include administration of the HDAC2 inhibitor before, simultaneously, substantially simultaneously, or after administration of the anti-neurodegenerative disease agent, but both agents will be administered in a regular sequence or order. The term “separate” means, unless otherwise specified, to keep apart one from the other. The term “simultaneously” means, unless otherwise specified, happening or done at the same time, i.e., the agents of the invention are administered at the same time. The term “substantially simultaneously” means that the agents are administered within minutes of each other (e.g., within 10 minutes of each other) and intends to embrace joint administration as well as consecutive administration, but if the administration is consecutive it is separated in time for only a short period (e.g., the time it would take a medical practitioner to administer two agents separately). As used herein, concurrent administration and substantially simultaneous administration are used interchangeably. Sequential administration refers to temporally separated administration of the agents described herein.

Combination therapy can also embrace the administration of the agents described herein (e.g., a HDAC2 inhibitor and an anti-neurodegenerative disease agent) in further combination with other biologically active ingredients (e.g., a different anti-neurodegenerative disease agent) and non-drug therapies (e.g., occupational therapy).

It should be appreciated that any combination of a HDAC2 inhibitor and another anti-neurodegenerative disease agent (e.g., an anti-neurodegenerative disease antibody) may be used in any sequence for treating a neurodegenerative disease. The combinations described herein may be selected on the basis of a number of factors, which include but are not limited to the effectiveness of inhibiting HDAC2, preserving cognitive function, reducing memory loss, reducing synaptic function, and/or alleviating at least one symptom associated with the neurodegenerative disease, or the effectiveness for mitigating the side effects of another agent of the combination. For example, a combined therapy described herein may reduce any of the side effects associated with each individual members of the combination, for example, a side effect associated with the anti-neurodegenerative disease agent.

In some embodiments, another anti-neurodegenerative disease agent is a medicinal therapy, a surgical therapy, and/or alternative therapy. Examples of the medicinal therapies include, but are not limited to, cholinesterase inhibitors (e.g., benztropine and trihexyphenidyl), levodopa, memantine, dopamine antagonists (e.g., pramipexole, ropinirole, rotigotine, and apomorphine), and MAO-B inhibitors (e.g., selegiline and rasagiline). Examples of a surgical therapy include, but are not limited to, deep brain stimulation, thalamotomy, pallidotomy, and subthalamotomy. Examples of alternative therapies include, but are not limited to music therapy, pet therapy, art therapy, occupational therapy, exercise, and occupational therapy.

### Kits for Use in Treating Neurodegenerative Disease

The present disclosure also provides kits for use in alleviating neurodegenerative disease. Such kits can include one or more containers comprising a HDAC2 inhibitor (e.g., a peptide inhibitor). In some embodiments, the HDAC2 inhibitor is any agent capable of reducing HDAC2 binding to Sp3 as described herein. In other embodiments, the kit comprises a HDAC2 inhibitor that is a small molecule inhibitor, an anti-HDAC2 antibody, or an agent that inhibits expression of HDAC2.

In some embodiments, the kit can comprise instructions for use in accordance with any of the methods described herein. The included instructions can comprise a description of administration of the HDAC2 inhibitors to treat, delay the onset, or alleviate a neurodegenerative disease according to any of the methods described herein. The kit may further comprise a description of selecting an individual suitable for treatment based on identifying whether that individual has a neurodegenerative disease. In still other embodiments, the instructions comprise a description of administering a HDAC2 inhibitor to an individual having, suspected of having, or at risk for a neurodegenerative disease.

The instructions relating to the use of a HDAC2 inhibitor generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating a neurodegenerative disease. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a HDAC2 inhibitor, such as a peptide inhibitor.

Kits may optionally provide additional components such as buffers and interpretive information. Normally, the kit comprises a container and a label or package insert(s) on or associated with the container. In some embodiments, the invention provides articles of manufacture comprising contents of the kits described above.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press;

Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### EXAMPLES

In order that the invention described herein may be more fully understood, the following examples are set forth. The examples described in this application are offered to illustrate the methods, compositions, and systems provided herein and are not to be construed in any way as limiting their scope.

### Materials and Methods

### Animal Models

All mouse work was approved by the Committee for Animal Care of the Division of Comparative Medicine at the Massachusetts Institute of Technology. Male CK-p25 mice were crossed with female CK or p25 mice to get WT, CK, p25 and double transgenic CK-p25 mice. CK or p25 mice were used as negative controls. 2.5-3.5 months old double transgenic CK-p25 mice (and their littermates) were used to induce p25 expression by changing food pellets containing doxycycline to ones lacking doxycycline. All behavioral experiments and ex vivo LTP recordings were performed between 6 and 8 weeks of p25 induction, the time when cognitive deficits are strongly observed.

### Behavioral Tests

Behavioral experiments were conducted blind. Fear Conditioning Test: For fear conditioning, mice were put in the conditioning chamber (TSE systems) for 3 min, followed by a 30 s auditory cue (3 kHz, 80 dB) after which a constant 2 s foot shock (0.8 mA) was applied. 24 hours later, mice were re-exposed to the training context for 3 minutes and their freezing behavior was scored for memory acquisition. 48 hours later, mice were habituated to a novel context for 2 min, followed by 2 min exposure to the auditory cue used for training (3 kHz, 80 dB), and their freezing behavior was scored for memory acquisition.

### Plasmid Construction

For shRNA plasmids, U6 promoter and shRNA sequences were introduced into pCDH vector (System Biosciences, CD511B-1) with the CMV promoter deleted. shRNA sequences and loop sequence are listed in Table 1. HDAC2 and Sp3 cDNA clones were purchased from TransOMIC, and subcloned into pCDH vector to express tagged proteins or chimera proteins using Gibson Assembly Master Mix (NEB, E2611S). shRNA-resistant mutants were generated using QuikChange II site-directed mutagenesis kit (Agilent Technologies). The primers used for the mutagenesis are listed in Table 2. These pCDH plasmids were used for expression in Neuro2A cells for co-immunoprecipitation as well as lentivirus preparations.

TABLE 1
List of shRNA sequences.

| | Sequence 5′ → 3′ | SEQ ID NO |
|---|---|---|
| Loop sequence | TTCAAGAGA | 2 |
| Control shRNA | AATTCTCCGAACGTGTCACG | 3 |
| HDAC2 shRNA | GGTCGTAGGAATGTTGCTGAT | 4 |
| Sp3 shRNA | GCACCTGTCCCAACTGTAAAG | 5 |
| Sap30 shRNA | GGAACAGAAGGAAGAGGAA | 6 |
| Ttrap shRNA | GCCATCAGGATTTCAAGTAAT | 7 |

TABLE 2
List of primers for mutagenesis.

| | | Primer Sequence 5′ → 3′ |
|---|---|---|
| HDAC2 shRNA- | Forward | GATGAAGGTGAAGGAGGCCGCAGAAACG |
| | | TGGCAGACCATAAGAAAGGAG |
| | | (SEQ ID NO: 8) |
| resistant | Reverse | CTCCTTTCTTATGGTCTGCCACGTTTCT |
| mutant | | GCGGCCTCCTTCACCTTCATC |
| | | (SEQ ID NO: 9) |
| Sp3 shRNA- | Forward | GTACCTCTCCCACCACCTTCCTTGCAAT |
| | | TCGGGCACGTACAAGCTACCCTCCGAAG |
| | | TCT |
| | | (SEQ ID NO: 10) |
| resistant | Reverse | AGACTTCGGAGGGTAGCTTGTACGTGCC |
| mutant | | CGAATTGCAAGGAAGGTGGTGGGAGAGG |
| | | TAC |
| | | (SEQ ID NO: 11) |

### Lentivirus Construction

HEK-293T cells were transfected with 7.5 μg lentivirus plasmid, 2.5 μg VSV-G, 1.9 μg pRSV-Rev and 5.0 μg pMDLg/pRRE using Lipofectamine 2000 (Life Technologies) according to the manufacturer's protocol. Next day, the media was exchanged with fresh media containing 20% FBS. Supernatant was collected 48 h later, centrifuged for 5 min at 300 g, sterile-filtered through a 0.45 μm filter, then centrifuged at 19,500 rpm for 2 h at 4° C. (Optima I-90K ultracentrifuge, SW41 Ti rotor) and discarded. The pellet was resuspended in cold Dulbecco's phosphate-buffered saline (DPBS, Life Technologies) overnight at 4° C., then aliquoted and stored at −80° C. The viral titer was estimated with the Lentivirus qPCR Titer kit (ABM Inc).

### Primary Cultured Neurons

Primary cortical neurons were dissociated from E15-16 Swiss-Webster embryos. The neurons were plated in 24-well plates (for RT-PCR) containing round coverslips (for mEPSC recordings), 6 cm dishes (for RNA-seq), or 10 cm dishes (for ChIP), all of which were coated with PDL (30 μg/mL, Sigma; P6407) and mouse laminin (2 μg/mL, Corning; 354232). The densities of cells were 1×10̂5 cells/mL/well for 24-well plate, 1.5×10̂6 cells/8 mL/dish for 6 cm dish and 4×10̂6 cells/15 mL/dish for 10 cm dish. Neurons were maintained with Neurobasal media supplemented with B27, penicillin/streptomycin and Glutamax (Life Technologies) and treated with 1 μM AraC at DIV5 to minimize glial cells. Half of media was changed with fresh media every 2-3 days. All experiments were performed using neurons at DIV17-22.

### Chromatin Immunoprecipitation (ChIP)

Crosslinking was performed with 1% formaldehyde at room temperature for Sp3 and acetylated histones. For HDAC2 ChIP, additional crosslinking with 2 mM disuccinimidyl glutarate (DSG) was done for 35 min followed by addition of formaldehyde (final 1%) and another 10 min incubation. The reaction was stopped with 125 mM glycine. For primary cultured neurons, cell pellets were lysed with 50 mM Hepes-KOH (pH 7.4), 140 mM NaCl, 1 mM EDTA, 10% glycerol, 0.5% NP-40, 0.25% TritonX-100, protease inhibitor cocktail for 10 min. Nuclei were pelleted by spinning at 1000 rpm for 5 min at 4° C. The pellets were resuspended with 10 mM Tris-HCl (pH8.0), 0.5 mM EGTA, 1 mM EDTA, 200 mM NaCl and rocked for 10 min at room temperature followed by centrifugation at 1000 rpm for 5 min at 4° C. The resultant pellets were nuclear fractions for ChIP experiments. For brain tissues, isolation of neuronal nuclei was conducted after crosslinking. Isolated nuclei were subjected to fluorescence-activated cell sorting (FACS) after staining with Alexa488-conjugated anti-NeuN antibody (Millipore, MAB 477X). Purified NeuN-positive nuclei or nuclear fractions of primary neurons were sonicated in 10 mM Tris-HCl (pH8.0), 0.5 mM EGTA, 1 mM EDTA, 0.5% (w/v) N-Lauroylsarcosine sodium salt using Bioruptor (setting high, 40 cycles of 30 s ON and 30 s OFF). Sheared chromatin was immunoprecipitated with antibodies against HDAC2 (Abcam; ab12169), Sp3 (Santa cruz; sc-644 X), or acetylated histone H4 (Active motif; 39925). Immunoprecipitated DNA was extracted by phenol/chloroform/isoamyl alcohol, purified by ethanol precipitation and subjected to quantitative PCR using primers specific to the promoter regions of the genes assayed (see Table 3 for primer sequences). The fluorescent signal of the amplified DNA (SYBR green, BioRad) was normalized to input.

TABLE 3
List of primers used in ChIP experiments. The
number after gene name indicates the
position of 3′ end of each primer from TSS.

| Gene name | Primer (5′-3′) | SEQ ID NO |
|---|---|---|
| Kcna2 −237 | CTACCCTCTCCCCTGTCTCC | 12 |
| Kcna2 −133 | GCAAAGAAAACACCCCATTC | 13 |
| Kcna2 +200 | AGTGTCCGGCATTCTGCT | 14 |
| Kcna2 +292 | CTCGCCCACCCAGACTAC | 15 |
| Grik2 −251 | TCAATCCTTGTCCCTTTTGC | 16 |
| Grik2 −339 | CAAGCAAGCACATCCACATC | 17 |
| Grik2 +317 | CAGGAAAGGAAGAGGGGAAC | 18 |
| Grik2 +228 | AGTGAGACAAAGCCCTCCAA | 19 |
| Digap1 −367 | GCTGAGATGTGGTTGGCTTT | 20 |
| Digap1 −270 | CCCCCAAGCCTATTCTGTTT | 21 |
| Digap1 +338 | GTGAATCAGGTGGGGACATC | 22 |
| Digap1 +419 | CAACAAGACCACAGGAAGCA | 23 |
| Lin7a −114 | TCTCCATCTGGCTACCAACC | 24 |
| Lin7a −22 | AGAGGGAAGACGGAAAGGAG | 25 |
| Lin7a +449 | AAGAGGGGCAGAGAAAGCTC | 26 |
| Lin7a +553 | GGGACAAACTTCCTCCCTTC | 27 |
| Kcnc3 −298 | TCGCTGTGCTGCTGAGTTAG | 28 |
| Kcnc3 −214 | CAGAAAGCTCAGGGATTGGA | 29 |
| Kcnc3 +435 | TTCGCCTACGTGCTCAACTA | 30 |
| Kcnc3 +541 | GTCTCGTCTATGCCCCAGAA | 31 |
| Gabbr2 −330 | AGCAGTACCCAACCACCTTG | 32 |
| Gabbr2 −433 | CTCCAGAGCCCCACGTTC | 33 |
| Gabbr2 +608 | GAGCTAGCCATCGAGCAGAT | 34 |
| Gabbr2 +529 | ACCTCGGTGTCGTAGAGTCG | 35 |
| Gabbr2 +4223 | CGCCCATAATCTACCTTTGC | 36 |
| Gabbr2 +4109 | GTGGGGGAAATTCCATGATA | 37 |
| Ogfrl1 −363 | AGACCGCAGGGATTCTAGGT | 38 |
| Ogrfl1 −465 | AGCCACAGCAGAAGACAAAAG | 39 |
| Ogrfl1 +203 | CCTCTTCAATGGGCAACCT | 40 |
| Ogrfl1 +116 | GAATCGGTCTGCCAGGTG | 41 |
| Nlgn1 −197 | AGTGGGCTTCAGCTCCTGTA | 42 |
| Nlgn1 −299 | GCCGCGTAGGTCTTCTTATG | 43 |
| Nlgn1 +413 | AAGCCGAGAGGAGTGAGACA | 44 |
| Nlgn1 +326 | CCGCTCGGAAGACTAGGAG | 45 |
| Scn3b −489 | TGTGCCACACCCTACCCTAT | 46 |
| Scn3b −410 | TGCCTTGATTAATGGGTTCC | 47 |
| Scn3b +260 | CACATTCTGTAGCCCAGACG | 48 |
| Scn3b +343 | CAGAATCTCGGGCTTCTACG | 49 |
| Scn3b +3906 | CAGTGTGCTTTCTCCCCTTC | 50 |
| Scn3b +4000 | AGAGGTTTGGGGCCTGTTAT | 51 |
| Syngr3 −201 | TGGGCCTCAGTTTCCTCTTA | 52 |
| Syngr3 −296 | CATAGCCAAGAGCATCGACA | 53 |
| Syngr3 +190 | AACGGACAGAAGGCAAAGTG | 54 |
| Syngr3 +104 | CAAAGCTCACGGGATCAAAG | 55 |
| Magi2 −263 | GAAGGGATGCAGCCTTGTTA | 56 |
| Magi2 −149 | TTGAGCCTTTTTGGTTTTCC | 57 |
| Magi2 +220 | AGAGAGAGCGAGCTGCAT | 58 |
| Magi2 +309 | TTGAAGCCAGACACAGCAAC | 59 |
| Synpr −294 | CCCTGACATTGGTGCTCTTT | 60 |
| Synpr −207 | TGGTTGGCAACAGTGGACTA | 61 |
| Synpr +162 | CTGAAGGGAACTGGTTCGAG | 62 |
| Synpr +246 | CCTGCCTGTCCTGTTCATTT | 63 |
| Cd81 +303 | ATTTCGTCTTCTGGGTGAGC | 64 |
| Cd81 +390 | CCTTCTCAGCAGGGCCTA | 65 |
| Mkrn1− 298 | CACTTCCATCAGCAGGGATT | 66 |
| Mkrn1 −400 | GGGGCTGTGTCTGCTCTTTA | 67 |
| Fam171b −358 | CCTCGGTGTCTAGTGGAAGG | 68 |
| Fam171b −250 | GCGTTTAGCTAGGCGGAGAT | 69 |
| Tanc2 +418 | CTGCCTCCGAATGAATGTG | 70 |
| Tanc2 +498 | AGACCAACCTCGGTGACAAC | 71 |
| Engase +343 | ATCTCGTTCTGGCAGTCTGG | 72 |
| Engase +436 | ACACGAACAGAAAGCCATCC | 73 |

### Gene Expression Analysis

RNA was extracted using RNeasy Plus Mini kit (QIAGEN). To ensure the quantitativity of reverse transcription (RT) and PCR reactions, 8-16 ng of RNA was used for each RT reaction with RNA to cDNA ECODRY™ Premix (double primed) (Clontech) and one fortieth of the RT product was used for each PCR reaction except the PCR for 28s rRNA, which was done using 1/240 of RT reaction as PCR template. The relative amount of RNA was calculated based on a standard curve of diluted control sample and normalized to that of 28s rRNA or HPRT. The comprehensive list of primers is shown in Table 3. For RNA-sequencing (RNA-seq), 300-500 ng of total RNA was used to prepare the library using TrueSeq total RNA Sample Prep Kit (Illumina). Sequencing of bar-coded libraries was conducted using the Illumina Hi-Seq 2000. Gene ontology analysis was done using DAVID Functional Annotation Tool.

TABLE 4
List of primers used in RT-qPCR experiments.

| Gene | Forward (5′-3′) | SEQ ID NO | Reverse (5′-3′) | SEQ ID NO |
|---|---|---|---|---|
| Hdac1 | GACGGCATTGACGACGAATC | 74 | TGAAGCAACCTAACCGGTCC | 90 |
| Hdac2 | TATGGGGAATACTTTCCTGG | 75 | TGACAGCATAGTATTTTCCC | 91 |
| Kcna2 | GCACCCACAAGACACCTATGA | 76 | GTCTCTGGGAACTGGGCTAAG | 92 |
| Grik2 | CAGTTGTGTATGACGACAGC | 77 | AGATTGTACCTTGATGGAGC | 93 |
| Digap1 | CCGAAGCTTGTCAACAAGAG | 78 | GTGTACCCTGACCATTCATC | 94 |
| Lin7a | GCTGCTATCAGTGAACGGAG | 79 | GCAGCCTTGAGAAGTTCCAC | 95 |
| Kcnc3 | TTTGAGGACCCCTACTCGTC | 80 | ATGAAGCCCTCGTGTGTCTC | 96 |
| Gabbr2 | TCAACGACACCATAAGGTTC | 81 | GGATGCTATACAGTGGAAGC | 97 |
| Ogfrl1 | AAGACTGGAAATGTTGCTCGG | 82 | GCTCGCCAAGGCTTTTAAGAA | 98 |
| Nlgn1 | TTTGCTAAAACTGGTGACCC | 83 | AAGCGGTTGGGTTTGGTATG | 99 |
| Scn3b | GATTGCTTCCCCTAGCTTCTCT | 84 | AGGAAATCTTTACCGCCCTCA | 100 |
| Syngr3 | ATGGAGGGAGCATCCTTTGG | 85 | CACCGCAATAGAAAACACCCA | 101 |
| Magi2 | CCCCAGGTTTCCGAGAAAAG | 86 | CCACCAATGATGGTAAACCC | 102 |
| Synpr | ACAGCCCTGTCATGTCCAGC | 87 | CAAATGTTTCCAGCCCAGAG | 103 |
| Hprt1 | TACCTAATCATTATGCCGAGGA | 88 | GAGCAAGTCTTTCAGTCCTG | 104 |
| 28s rRNA | TCATCAGACCCCAGAAAAGG | 89 | GATTCGGCAGGTGAGTTGTT | 105 |

### Immunoblotting

Brain tissues or cell pellets were lysed in 50 mM Tris-HCl (pH8.0), 150 mM NaCl, 1 mM EDTA, 1% NP-40, and complete protease inhibitor cocktail (Roche) with 20 strokes using the Dounce tight homogenizer. After centrifugation at 10000×g twice for 10 min, supernatants were subjected to co-immunoprecipitation or western blot analysis (Bio-rad). Two micrograms of anti-HDAC2 antibody (ab12169) or 15 μL of anti-Flag M2 affinity gels (Sigma) were used for immunoprecipitations. The antibodies used for immunoblotting were anti-HDAC2 (1 μg/mL, ab12169), anti-Sp3 (1 μg/mL, sc-644 X), anti-Sin3A (1 μg/mL, Abcam; ab3479), and anti-λ-tubulin (1:500, Sigma; F2043).

### Immunohistochemistry

Mice were anesthetized with isoflurane and transcardially perfused with 4% paraformaldehyde. Brains were coronally sectioned at 40 μm with a vibratome (Leica). The sections were stained with anti-Sp3 (1:1000, sc-644 X) antibody. copGFP signals were detected without staining.

### Stereotaxic Injections

One microliter of lenti-virus expressing either shRNA or mCherry-fusion protein constructs was stereotaxically injected into dorsal hippocampal area CA1 of both hemispheres at 0.1 μL/min. Injection needles were left in place 2 min before and 5 min after injection to assure even distribution of the virus. Injections were performed 4 weeks before LTP recordings or behavioral tests. The coordinates of injection sites for LTP recordings were anterior-posterior position (AP) −2.3 mm, medial-lateral position (ML) ±1.35 mm from Bregma, dorsoventral (DV) −1.35 mm from cortical surface). For behavioral tests, the viruses were injected into two more sites, AP: −1.70 mm, ML: ±1.66 mm, DV: −1.27 mm, in addition to the sites described above to cover the entire dorsal hippocampal CA1 area. All infusion surgeries were performed under aseptic conditions and anesthesia (ketamine/xylazine) in accordance with the Massachusetts Institute of Technology's Division of Comparative Medicine guidelines.

### Electrophysiology

Acute hippocampal slices were prepared from the mice injected with lenti-virus, 4 weeks after viral injection. The mice were anesthetized with isoflurane and decapitated. The experimenter was blinded to which virus was injected. Transverse hippocampal slices (400 μm thick) were prepared in ice-cold dissection buffer (211 mM sucrose, 3.3 mM KCl, 1.3 mM NaH₂PO4, 0.5 mM CaCl₂, 10 mM MgCl₂, 26 mM NaHCO₃ and 11 mM glucose) using a Leica VT1000S vibratome (Leica). Slices were recovered in a submerged chamber with 95% O₂/5% CO₂-saturated artificial cerebrospinal fluid (ACSF) consisting of 124 mM NaCl, 3.3 mM KCl, 1.3 mM NaH₂PO₄, 2.5 mM CaCl₂, 1.5 mM MgCl₂, 26 mM NaHCO₃ and 11 mM glucose for 1 h at 28-30° C. To ensure that an equivalent number of virus-transduced cells were present in each slice, the number of GFP/mCherry expressing cells was quantified. For extracellular recording, CA1 field potentials evoked by Schaffer collateral stimulation with bipolar electrode was measured every 30s. After recording the baseline for 15 min, LTP was induced by repeated (2 times) theta-burst stimulations (TBS, containing 10 brief bursts which consisted of four pulses at 100 Hz). The slopes of fEPSPs were measured to quantify the strength of synaptic transmission. HEKA instrument (EPC10) was used for data acquisition and data were analyzed with pClamp10 (Axon Instruments). The input-output curve was obtained by plotting the slopes of fEPSPs against stimulation intensity (mA). For mEPSC recordings of primary cortical neurons (DIV17-22), the external solution consisted of 140 mM NaCl, 4 mM KCl, 2 mM CaCl₂, 2 mM MgCl₂, 10 mM HEPES, and 10 mM glucose (pH 7.3 with NaOH), 315 mOsm. The internal solution contained 145 mM CsCl, 5 mM NaCl, 10 mM HEPES, 10 mM EGTA, 4 mM Mg-ATP, and 0.3 mM Na₂-GTP (pH 7.3 with CsOH), 305 mOsm. The external solution also contained 1 μM TTX, 10 μM bicuculline. Series resistance was compensated. The membrane potential of each cell was patched at −70 mV during recording. Recordings were obtained at room temperature. Data were acquired using the Axopatch 200B amplifier and analyzed with the pClampl0 software (Molecular Devices).

### Bioinformatics

Weighted gene co-expression network analysis was performed with an available R-package (labs.genetics.ucla.edu/horvath/CoexpressionNetwork/, labs.genetics.ucla.edu/horvath/CoexpressionNetwork/Rpackages//#technicalReports). The dataset for gene expression from the cerebral cortex of 187 healthy individuals was drawn from GSE15222 in Gene Expression Omnibus (GEO; ncbi.nlm.nih.gov/geo/). The dataset of hippocampal gene expression in AD patients and controls was from GSE5281. For RNA-Seq data, single-end sequencing reads were mapped to mouse genome assembly (mm9) using Tophat2. Differential expression analysis was performed using Cuffdiff module of Cufflinks. Significantly altered genes were the genes with adjusted P-value less than 0.05 between two groups. RNA-Seq signals at HDAC2 and Sp3 loci were visualized using IGV browser. Synapse genes were obtained from SynSysNet (bioinformatics.charite.de/synsysnet/). Gene ontology was assessed using DAVID web servers. RNA-Seq datasets of an Alzheimer's mouse model, CK-p25 were also used for overlap analysis. Software R was used for generating the plots unless specified. Following each genetic perturbation (HDAC2 or Sp3 KD), genes were classified into three groups: up-regulated, down-regulated, and un-changed. For given two groups (one from HDAC2 KD, one from Sp3 KD), overlap counts were calculated, and the statistical P-values were generated by Fisher's exact test in R.

### Statistics

Student's or Welch's t-test was used for the statistical comparison of two groups, following f-test. Multiple comparisons were carried out with Dunnett's test unless otherwise noted. To examine the significance of overlaps in RNA-seq data, the Fishers' exact test was used.

### Example 1: Identification of Potential HDAC2 Co-Regulators Through WGCNA

HDACs, including HDAC2, associate with a number of different chromatin-modifying complexes, each of which regulates multiple processes within cells. To determine which binding partners are essential for HDAC2 recruitment to genes involved in particular processes, techniques other than classical immuno-precipitation (IP) followed by mass spectrometry (mass spec) were considered. IP-mass spec would indiscriminately identify all proteins bound to HDAC2 and would be of limited value in pinpointing the specific proteins that mediate the recruitment of HDAC2 to genes involved in synaptic plasticity. Due to these caveats, weighted gene co-expression network analysis (WGCNA) was utilized. Under the hypothesis that genes with similar expression patterns often encode for interacting proteins or groups of proteins involved in similar cellular processes, WGCNA was applied to publicly available gene expression data from 187 healthy human post-mortem brains.

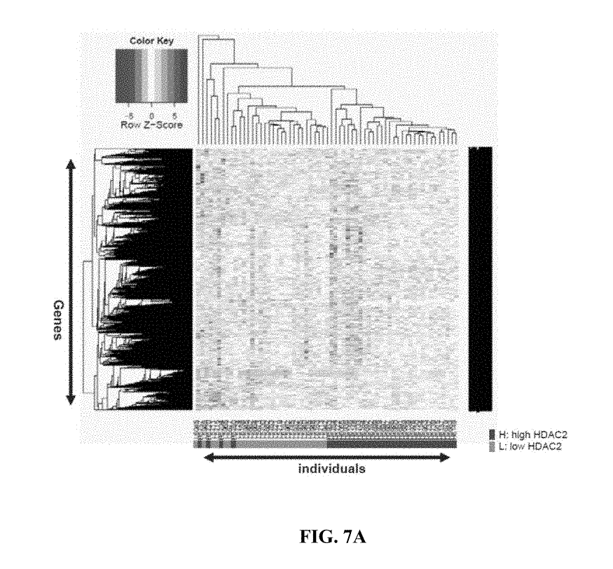

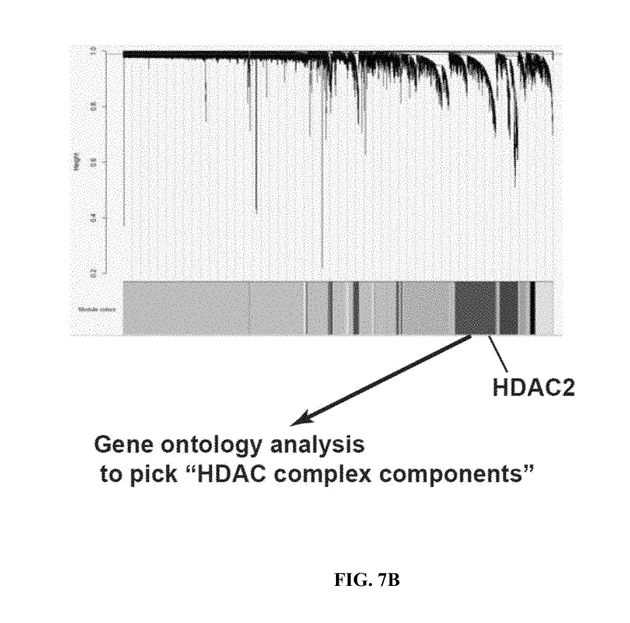

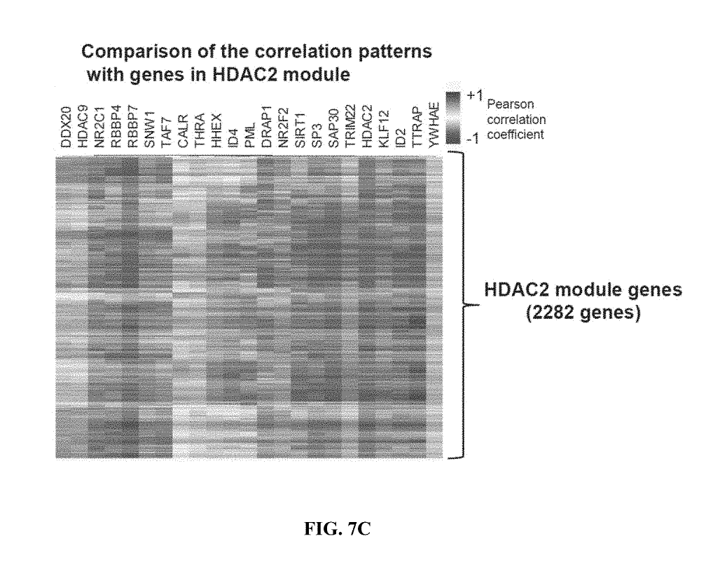

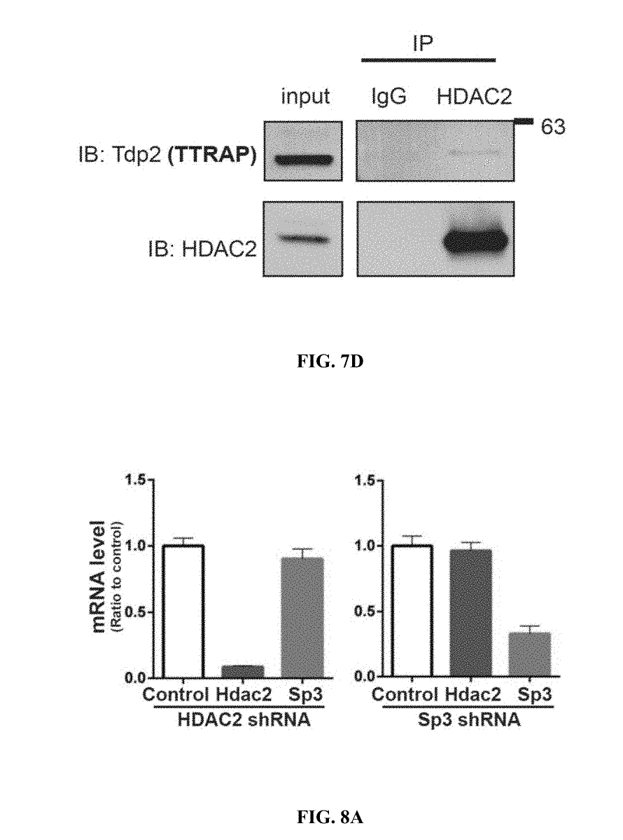

As a pilot study, a subset of 28 individuals with “high” HDAC2 expression (greater than one standard deviation above the mean) and 35 with “low” HDAC2 expression (greater than one standard deviation below the mean) was extracted and unbiased clustering of global gene expression was then performed (FIG. 7A). With few exceptions, this analysis reliably distinguished “high” from “low” HDAC2 expressing individuals, indicating that a gene expression signature can be associated with HDAC2 levels.

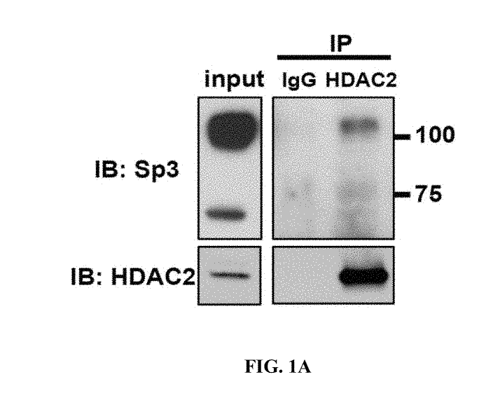

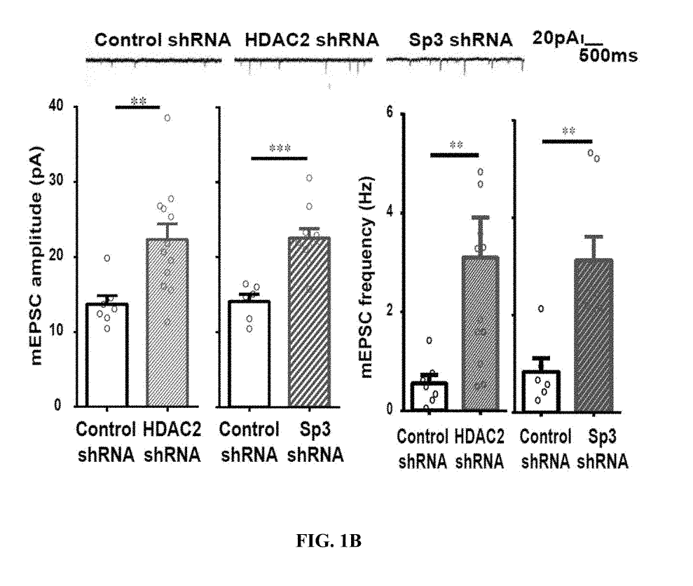

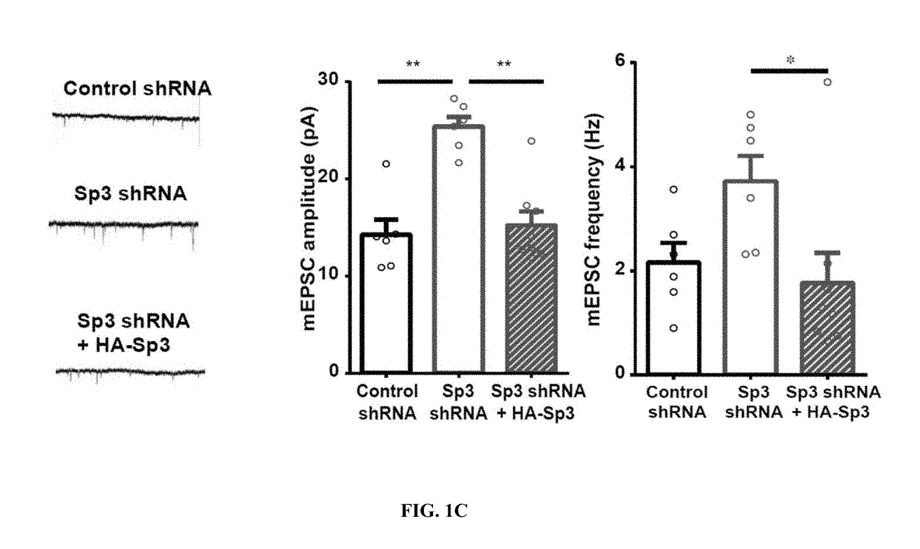

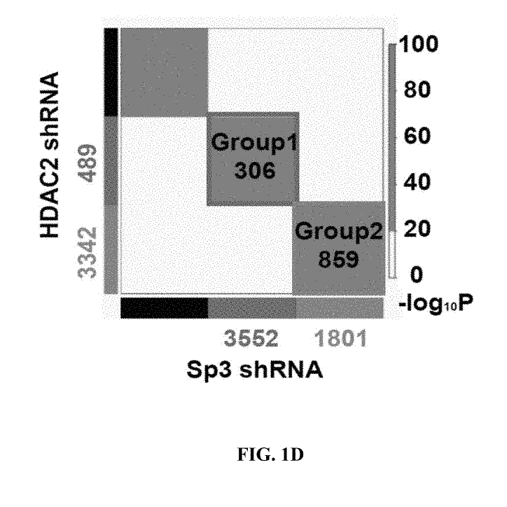

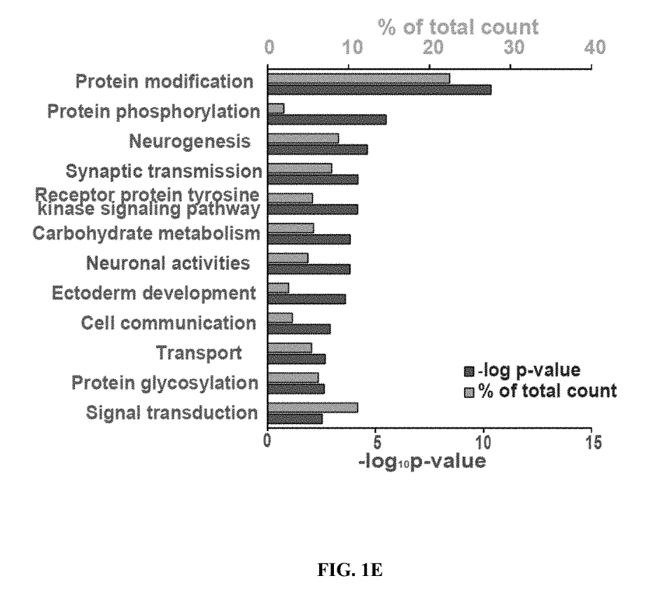

Next, whether this natural variation in HDAC2 gene expression could be employed to identify the HDAC2 binding partners involved in synaptic plasticity was tested. Therefore, WGCNA was performed on the entire dataset (irrespective of HDAC2 levels) and genes most tightly correlated or anti-correlated with HDAC2 based on gene expression were identified (FIG. 7B). This analysis revealed an HDAC2-containing module of 2,282 genes, which included many genes encoding for known HDAC2 binding proteins. Based on gene ontology (GO) analysis, the list of potential HDAC2 co-regulators was further narrowed down to transcriptional repressors (as defined by the GO terms “histone deacetylase binding”, “transcription corepressor activity”, “histone deacetylase activity” and “transcription repressor activity”). Finally, the pairwise correlation between the transcriptional repressors (including HDAC2) and genes in the HDAC2-module was calculated to find the putative HDAC2 co-regulators showing the same direction of correlation as HDAC2 (FIG. 7C). The consequent list of 22 candidates included several genes encoding HDAC2 binding proteins as previously reported, such as the DNA-binding proteins, Sp3, Tdp2 and Sap30. The physical interaction of Sp3 and Tdp2 to HDAC2 was confirmed through immunoprecipitation of HDAC2 followed by Western blotting using anti-Sp3 and anti-Tdp2 antibodies (FIGS. 1A and 7D).

### Example 2: Sp3 Negatively Regulates Synaptic Function

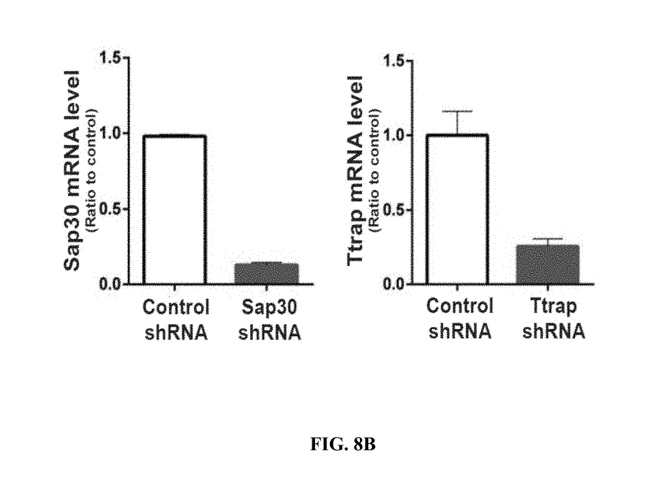

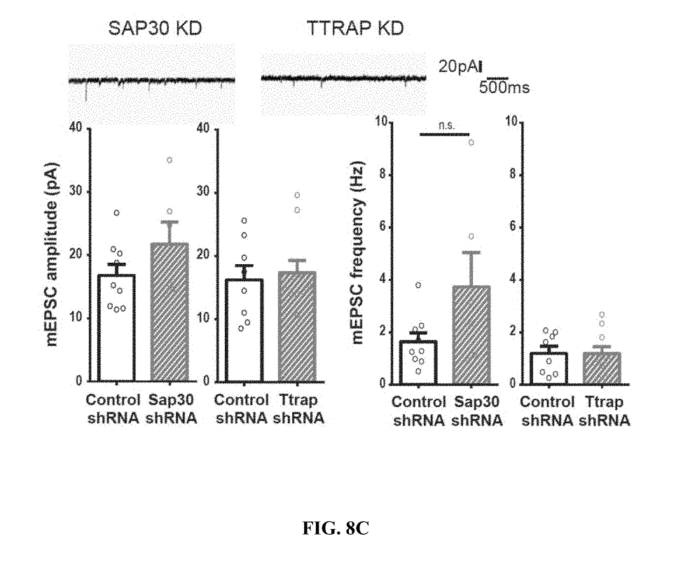

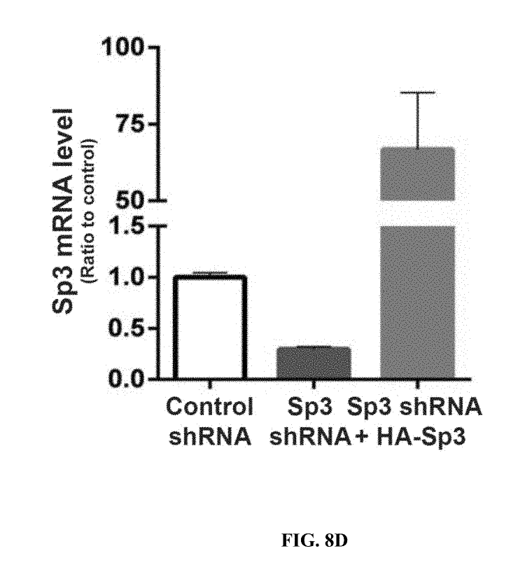

HDACs, including HDAC2, cannot directly bind DNA so subsequent efforts were focused on identifying HDAC2 interacting proteins that can bind DNA (Sp3, Sap30 and Ttrap/Tdp2). To aid in identifying whether these three proteins could be required for recruitment of HDAC2 to synaptic genes, the role of each protein in regulating synaptic function was assessed. Miniature excitatory post-synaptic currents (mEPSCs) were measured from cultured mouse primary neurons transduced with shRNA targeting HDAC2, Sp3, Sap30 or Ttrap (transduction with each shRNA resulted in greater than 50% reduction of mRNA; FIGS. 8A-8B). As expected, HDAC2 knockdown resulted in increased mEPSC amplitude and frequency (FIG. 1B). Interestingly, knockdown of Sp3 increased average mEPSC amplitude and frequency (FIG. 1B), while knockdown of Sap30 or Ttrap did not significantly alter either parameter (FIG. 8C). This facilitation of mEPSCs by Sp3 knockdown was completely reversed by expression of an shRNA-resistant form of Sp3, confirming the specificity of the effect (FIGS. 1C and 8D).

### Example 3: Sp3 Represses the Expression of Synaptic Genes Via the Recruitment of HDAC2

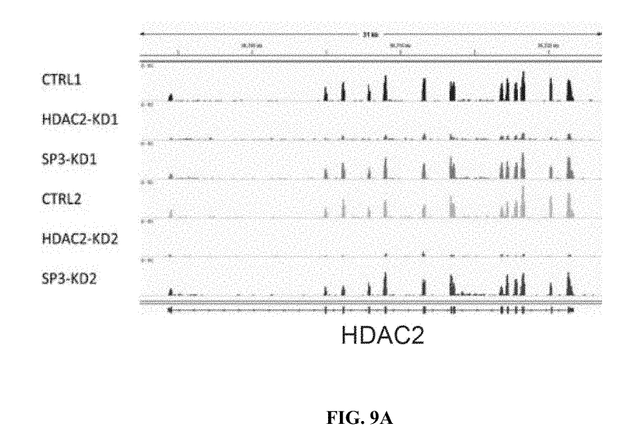

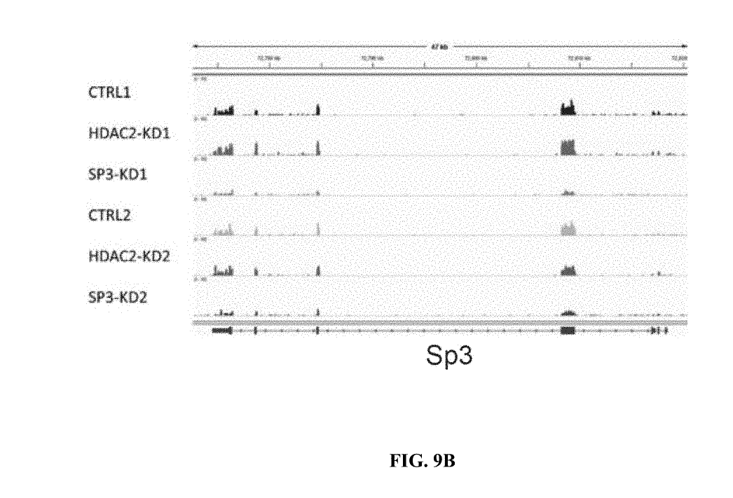

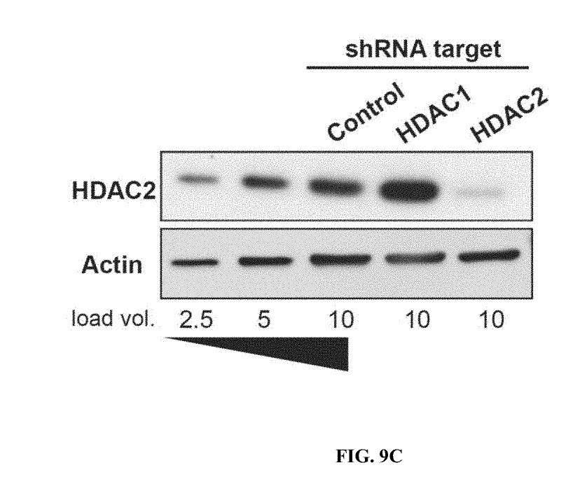

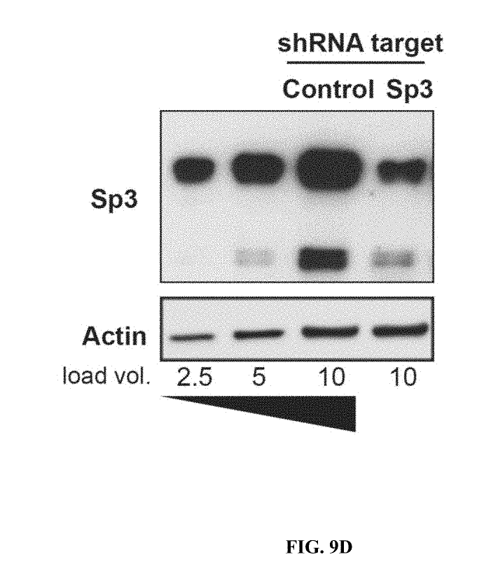

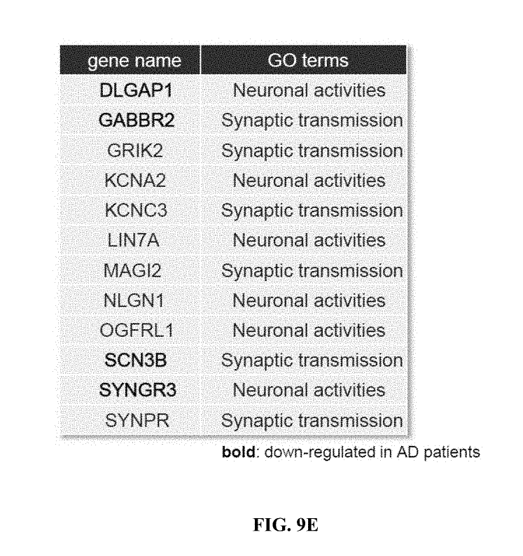

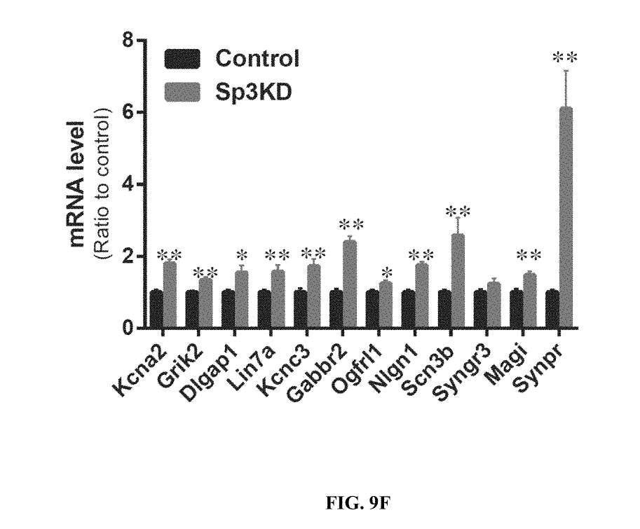

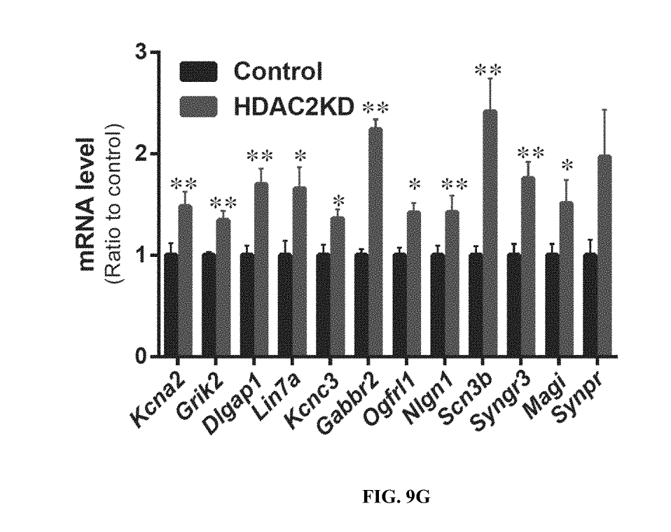

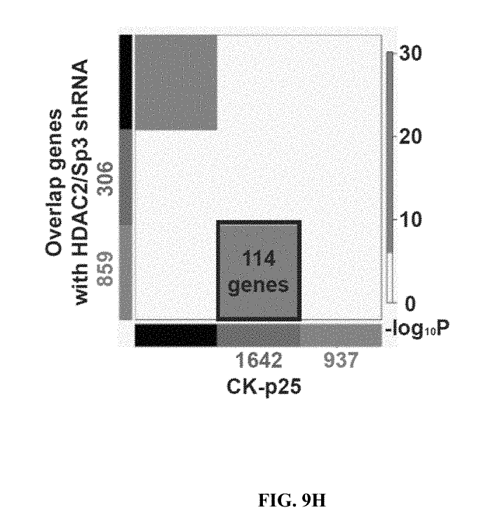

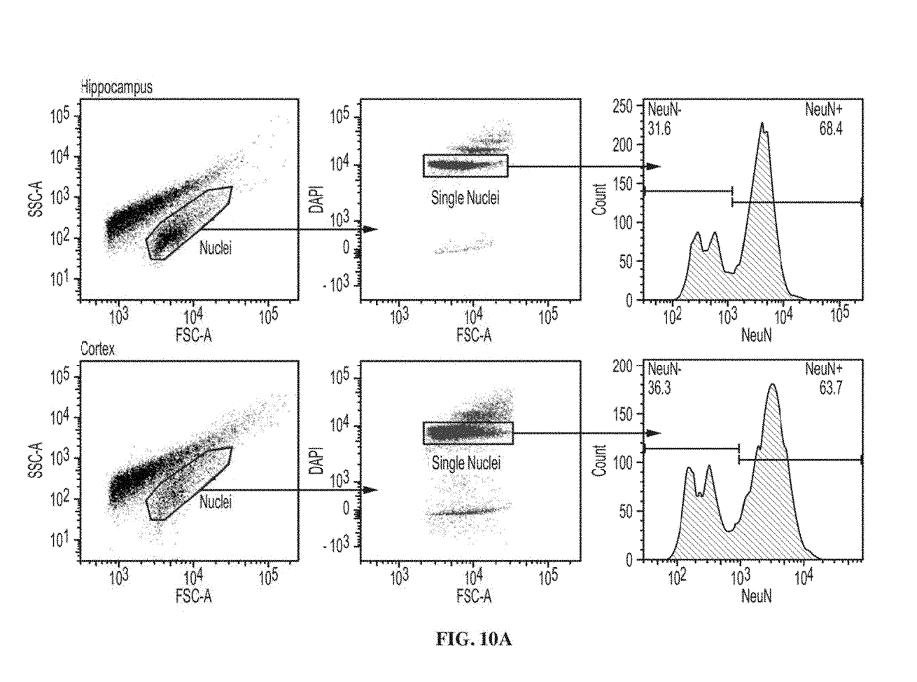

Since Sp3 binds to HDAC2 and depletion of Sp3 from mouse primary neurons recapitulated the effect of HDAC2 knockdown on mEPSCs, whether Sp3 and HDAC2 co-regulate synaptic gene expression in neurons was determined. To do so, transcriptomic analysis through RNA-sequencing (RNA-seq) from primary neurons transduced with control, HDAC2 or Sp3 shRNA (with >50% reduction of each protein; FIGS. 9A-9D) was performed. A statistically significant overlap of genes altered by knockdown of HDAC2 or Sp3 was found supporting that HDAC2 and Sp3 are functionally similar (FIG. 1D). Intriguingly, genes involved in synaptic transmission and neuronal activities were significantly enriched among the genes up-regulated after knockdown of either HDAC2 or Sp3 (FIG. 1E). A number of these changes in gene expression were validated by reverse transcription followed by quantitative PCR (RT-qPCR) including changes in the expression of subunits of potassium channels, sodium channels, and synaptic membrane proteins and receptors (FIGS. 9E-9G).

To examine if the genes co-regulated by HDAC2 and Sp3 are changed under pathological conditions, the overlapping genes altered by HDAC2 or Sp3 knockdown with the genes dysregulated in the CK-p25 mouse model of neurodegeneration, which displays elevated levels of HDAC2 in the hippocampus, was compared. In addition, these mice exhibit memory deficits and several AD-related pathologies such as neuronal loss, Tau hyperphosphorylation, Tau aggregation, increased amyloid load, and reduced synaptic density, following 6-week induction of p25 by withdrawing doxycycline. p25, a truncated version of p35, is an activator of cyclin-dependent kinase 5 (CDK5) and is implicated in AD. Inhibition of p25 generation prevents the expression of AD phenotypes in an AD model mice, supporting the notion that p25 accumulation can be a trigger of AD. Accordingly, gene expression and epigenomic signatures of the CK-p25 mouse after p25 induction correlate with those of human AD patients.

Interestingly, genes up-regulated by HDAC2 or Sp3 knockdown showed significant overlap with genes down-regulated in CK-p25 mice (FIG. 9H), as well as genes down-regulated in the brains of AD patients (Table 5). Specifically, synaptic genes like Dlgapl, Gabbr2, Scn3b, and Syngr3 are down-regulated in both CK-p25 mice and AD patients, and negatively co-regulated by HDAC2 and Sp3. Overall, the genome-wide expression analysis provided evidence that Sp3 and HDAC2 negatively regulate the expression of an overlapping set of genes related to synaptic function.

TABLE 5
Enrichment of genes up-regulated by HDAC2/Sp3
knockdown for terms in the CGP
database (broadinstitute.org/gsea/msigdb/annotate.jsp.)

| Gene Set Name | Description | FDR |
|---|---|---|
| BLALOCK_ALZHEIMERS_ | Genes down-regulated | 8.39E− |
| DISEASE_DN | in brain from patients | 30 |
| | with Alzheimer's disease. | |
| GRAESSMANN_APOPTOSIS_ | Genes down-regulated | 8.87E− |
| BY_DOXORUBICIN_DN | in ME-A cells (breast | |
| | cancer) undergoing | |
| | apoptosis in response to | |
| | doxorubicin | |
| | [PubChem = 31703]. | |
| GOBERT_ | Genes down-regulated | 4.83E− |
| OLIGODENDROCYTE_ | during differentiation of Oli- | |
| DIFFERENTIATION__DN | Neu cells (oligodendroglial | |
| | precursor) in response | |
| | to PD174265 | |
| | [PubChemID = 4709]. | |
| NUYTTEN_EZH2_ | Genes up-regulated in PC3 | 1.64E− |
| TARGETS_UP | cells (prostate cancer) | |
| | after knockdown of EZH2 | |
| | [GeneID = 2146] by RNAi. | |
| WONG_ADULT_TISSUE_ | The ‘adult tissue stem’ | 1.97E− |
| STEM_MODULE | module: genes | |
| | coordinately up-regulated | |
| | in a compendium of | |
| | adult tissue stem cells. | |
| SCHAEFFER_PROSTATE_ | Genes down-regulated in | 6.76E− |
| DEVELOPMENT_48HR_DN | the urogenital sinus (UGS) | |
| | of day E16 females | |
| | exposed to the androgen | |
| | dihydrotestosterone | |
| | [PubChem = 10635] | |
| | for 48 h. | |
| GEORGES_TARGETS_ | Genes down-regulated in | 3.06E− |
| OF_MIR192_AND_MIR215 | HCT116 cells (colon | |
| | cancer) by expression of | |
| | MIR192 or MIR215 | |
| | [GeneID = 406967; | |
| | 406997] at 24 h. | |
| BENPORATH_ | Set ‘Suz12 targets’: genes | 9.83E− |
| SUZ12_TARGETS | identified by ChIP on chip | |
| | as targets of the Polycomb | |
| | protein SUZ12 | |
| | [GeneID = 23512] in human | |
| | embryonic stem cells. | |
| PEREZ_TP53_TARGETS | Genes up-regulated in the | 2.60E− |
| | HMEC cells (primary | 18 |
| | mammary epithelium) upon | |
| | expression of TP53 | |

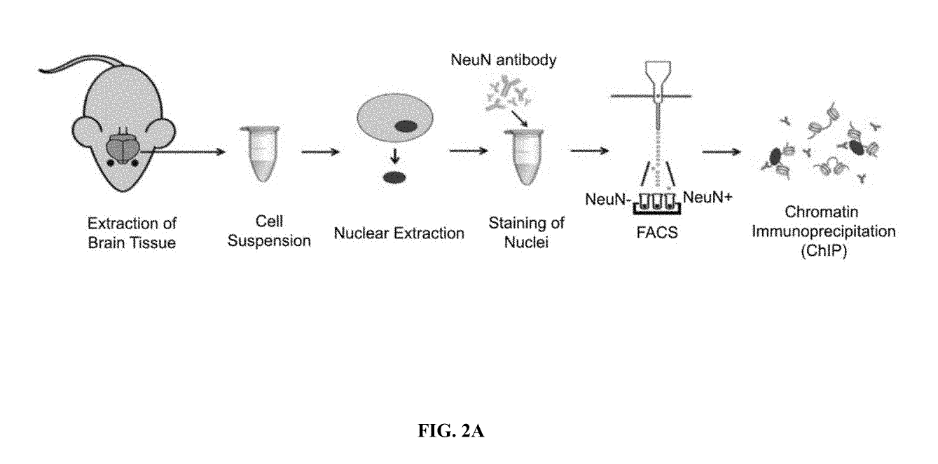

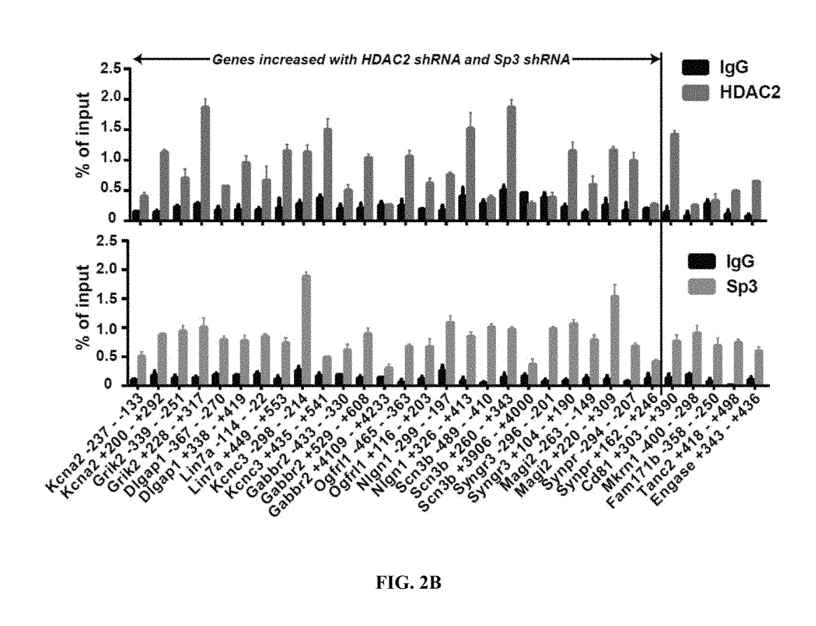

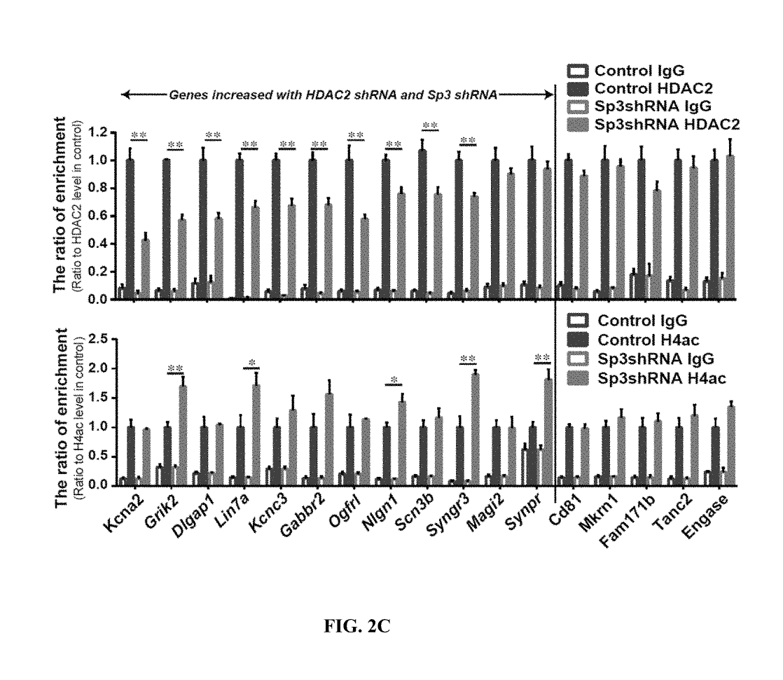

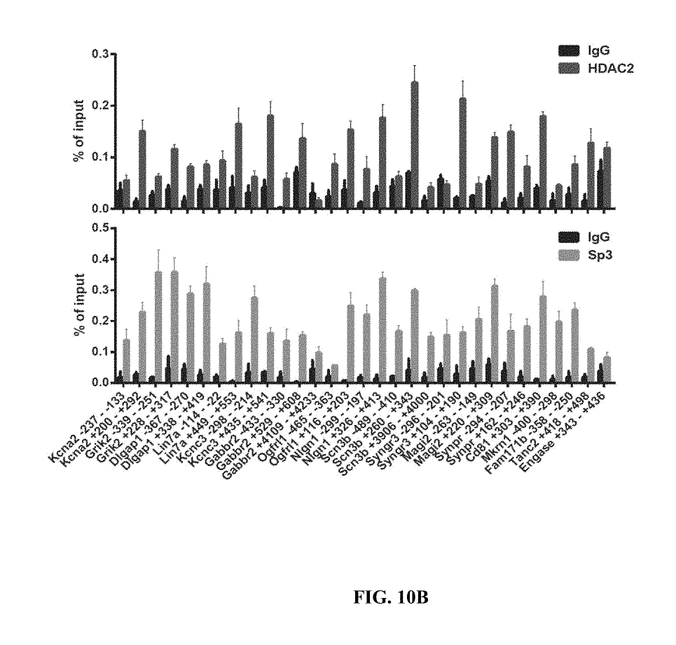

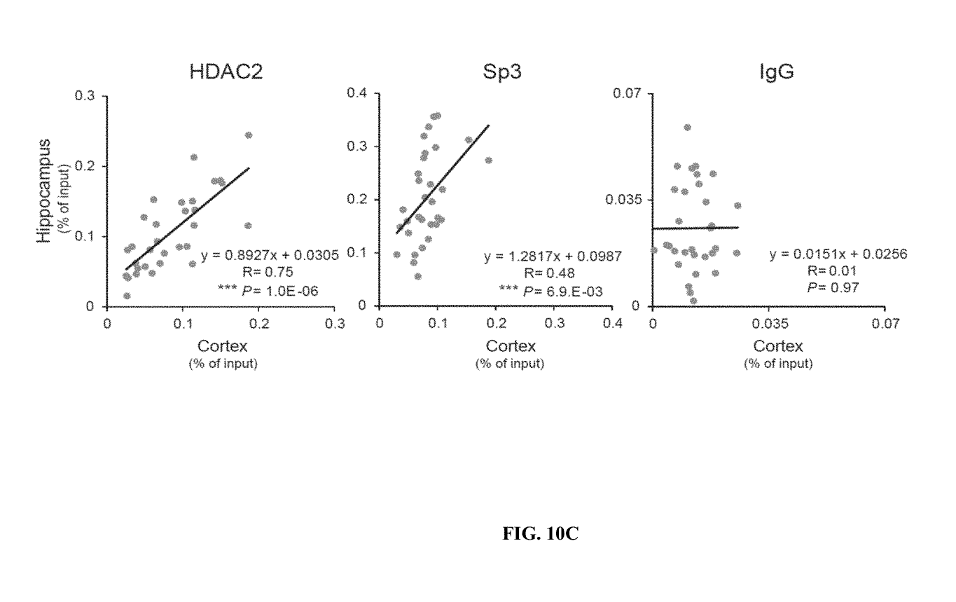

Taken together, these findings support the notion that the DNA-binding protein, Sp3, may serve to recruit HDAC2 to the promoters of genes involved in synaptic function. To address this hypothesis, chromatin immunoprecipitation (ChIP) followed by qPCR (ChIP-qPCR) was utilized to determine whether HDAC2 and Sp3 directly bind to the promoters of synaptic genes that were up-regulated after HDAC2 or Sp3 knockdown (FIG. 9E). Primer pairs were designed to amplify regions of the promoter both upstream and downstream of the transcription start site (TSS). Additional primers amplify regions roughly 4 kb downstream of the TSS and serve as negative controls for HDAC2 and Sp3 enrichment, as these proteins have previously been shown to localize to promoter regions. Due to interest in the role of HDAC2 and Sp3 at the promoters of synaptic genes and in neuronal function, neurons from the mouse brain were isolated and directly probed. Isolation of neuronal nuclei was achieved through staining for the neuronal marker, NeuN, followed by fluorescence-activated cell sorting (FACS) to separate NeuN-glial populations from NeuN+ neurons (FIGS. 2A and 10A). ChIP-qPCR using chromatin derived from cortical neuronal (NeuN+) nuclei of wild-type mice with anti-HDAC2 and anti-Sp3 antibodies demonstrated that HDAC2 and Sp3 colocalized at the promoters of synaptic genes, with clear enrichment relative to the IgG control (FIGS. 2B-2C). In ChIP-qPCR experiments using NeuN+ nuclei derived from hippocampal tissue, the enrichment and distribution of HDAC2 and Sp3 at synaptic gene promoters was similar to that observed in cortical neurons, suggesting that this phenomenon is conserved across brain regions (FIGS. 10B-10C).

Next, whether Sp3 mediates HDAC2 recruitment to the promoters of synaptic genes co-regulated by Sp3 and HDAC2 was tested. To address this question, the effect of Sp3 knockdown on HDAC2 enrichment at synaptic gene promoters in primary neurons was examined. Interestingly, ChIP experiments revealed that knockdown of Sp3 alone was sufficient to significantly reduce HDAC2 recruitment to the promoters of these genes (FIG. 2D). Importantly, HDAC2 enrichment at control genes (Cd81, Mkrnl, Fam171b, Tanc2, Engase), defined by a lack of change in expression after knockdown of HDAC2 or Sp3, was not affected by loss of Sp3 (FIG. 2D). Whether histone H4 acetylation at co-regulated synaptic gene promoters was altered by Sp3 knockdown was tested, as would be expected if HDAC2 recruitment to these sites was reduced. Indeed, the decrease in HDAC2 binding due to knockdown of Sp3 was accompanied by increased histone H4 acetylation at the promoters of several genes including Grik2, Lin7a, Nlgnl, Syngr3 and Synpr (FIG. 2E). These findings are consistent with the idea that Sp3 recruits HDAC2 to the promoters of synaptic genes where HDAC2 then mediates the deacetylation of histones to regulate gene expression.

### Example 4: Expression of HDAC2 and Sp3 are Deregulated in AD

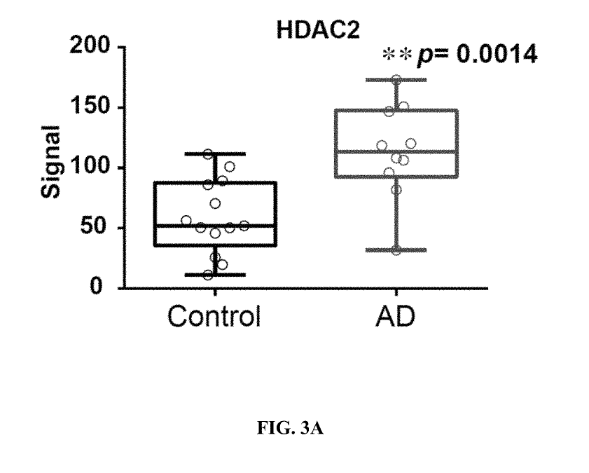

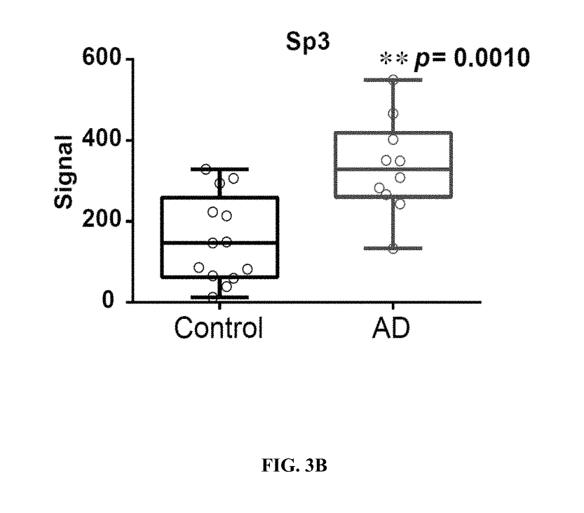

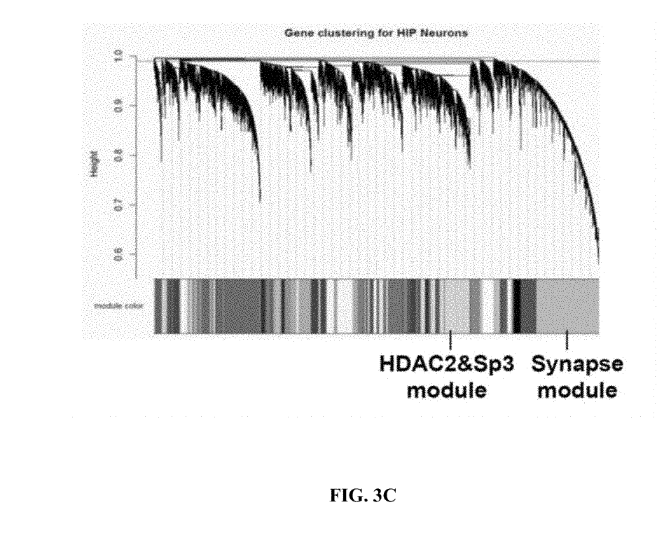

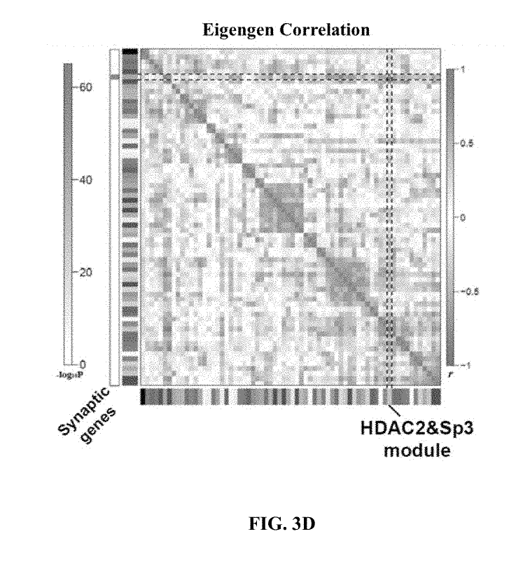

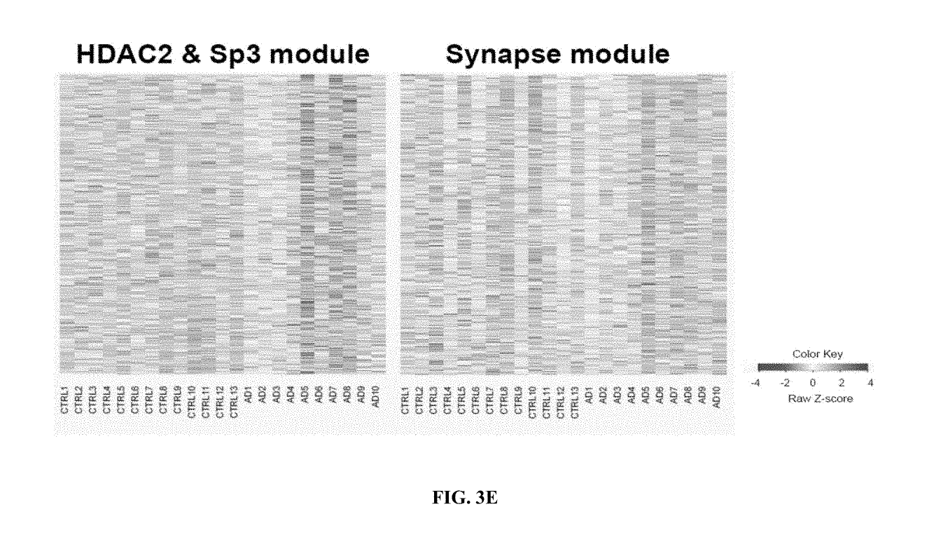

Gene expression profiling indicated that HDAC2 and Sp3 co-regulate a subset of synaptic genes, many of which are also deregulated in the context of AD pathology. These observations, together with earlier findings that HDAC2 protein levels were increased in AD patients and mouse models of neurodegeneration, prompted testing whether Sp3 expression might also be upregulated in AD. First, published gene expression data collected from hippocampal CA1 pyramidal neurons from 13 healthy controls and 10 AD patients was examined and significant increases in the expression of both HDAC2 and Sp3 in AD patients was found (FIGS. 3A-3B and Table 6). Furthermore, WGCNA was applied to the dataset to investigate the alteration of gene expression networks in AD patients. Even in this dataset combining healthy controls and AD patients, it was observed that HDAC2 and Sp3 segregated into the same gene expression module (FIG. 3C). Moreover, the expression of genes in the HDAC2/Sp3 module was higher in AD patients compared with controls, and negatively correlated with the expression of genes in the module most enriched for synaptic function (FIGS. 3D-3E).

TABLE 6
Human tissue information for control subjects and AD patients.

| GEO | | Disease | | |
|---|---|---|---|---|
| Accession: | Sample Name: | State: | Sex: | Age: |
| GSM119628 | HIP control 1 | normal | male | 85 days |
| GSM119629 | HIP control 2 | normal | male | 80 years |
| GSM119630 | HIP control 3 | normal | male | 80 years |
| GSM119631 | HIP control 4 | normal | female | 102 years |
| GSM119632 | HIP control 5 | normal | male | 63 years |
| GSM119633 | HIP control 6 | normal | male | 79 years |
| GSM119634 | HIP control 7 | normal | male | 76 years |
| GSM119635 | HIP control 8 | normal | male | 83 years |
| GSM119636 | HIP control 9 | normal | male | 79 years |
| GSM119637 | HIP control 10 | normal | female | 88 years |
| GSM119638 | HIP control 11 | normal | female | 73 years |
| GSM119639 | HIP control 12 | normal | male | 69 years |
| GSM119640 | HIP control 13 | normal | male | 78 years |
| GSM238799 | HIP_affected_1 | Alzheimer's | female | 73 years |
| | | Disease | | |
| GSM238800 | HIP_affected_2 | Alzheimer's | male | 81 years |
| | | Disease | | |
| GSM238801 | HIP_affected_3 | Alzheimer's | male | 78 years |
| | | Disease | | |
| GSM238802 | HIP_affected_4 | Alzheimer's | male | 75 years |
| | | Disease | | |
| G5M238803 | HIP_affected_5 | Alzheimer's | female | 70.8 years |
| | | Disease | | |
| GSM238804 | HIP_affected_6 | Alzheimer's | female | 85 years |
| | | Disease | | |
| GSM238805 | HIP_affected_7 | Alzheimer's | female | 77 years |
| | | Disease | | |
| GSM238806 | HIP_affected_8 | Alzheimer's | male | 79 years |
| | | Disease | | |
| GSM238807 | HIP_affected_9 | Alzheimer's | male | 88 years |
| | | Disease | | |
| GSM238808 | HIP_affected_10 | Alzheimer's | male | 72 years |
| | | Disease | | |

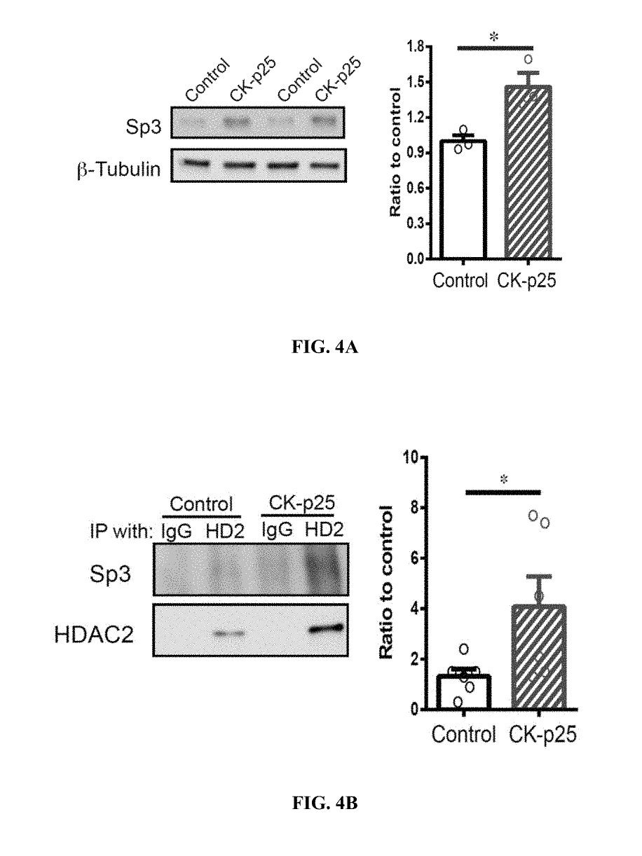

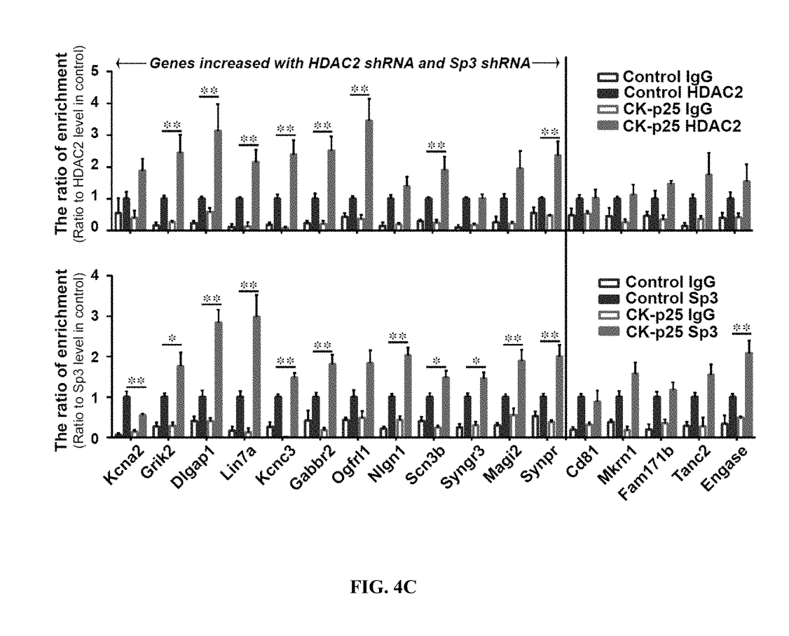

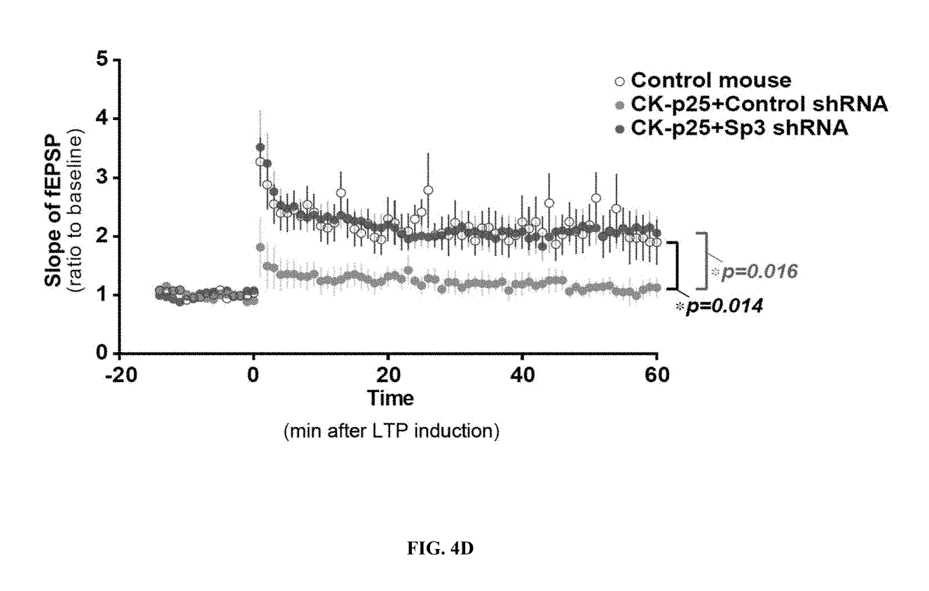

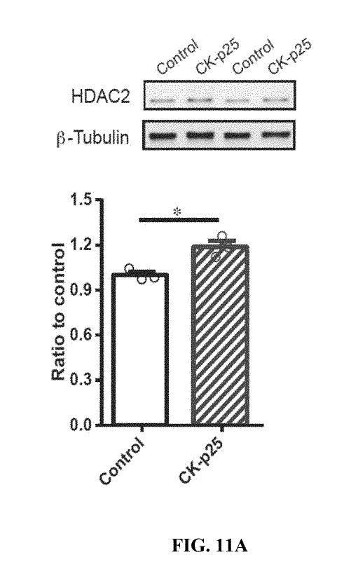

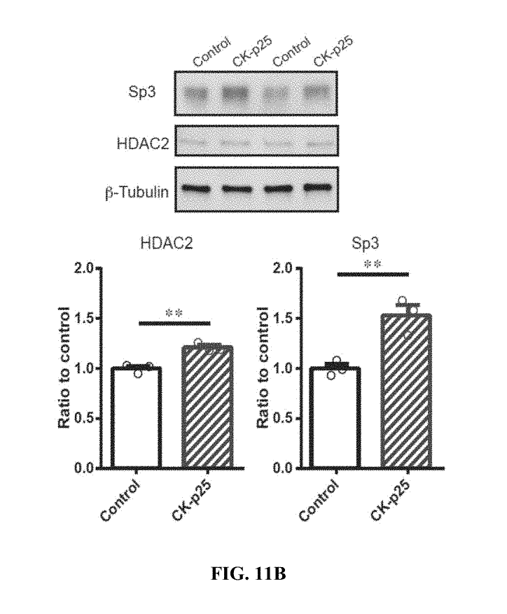

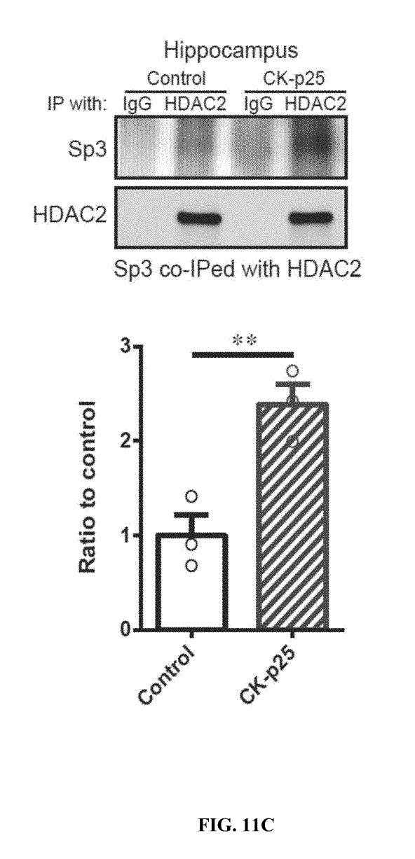

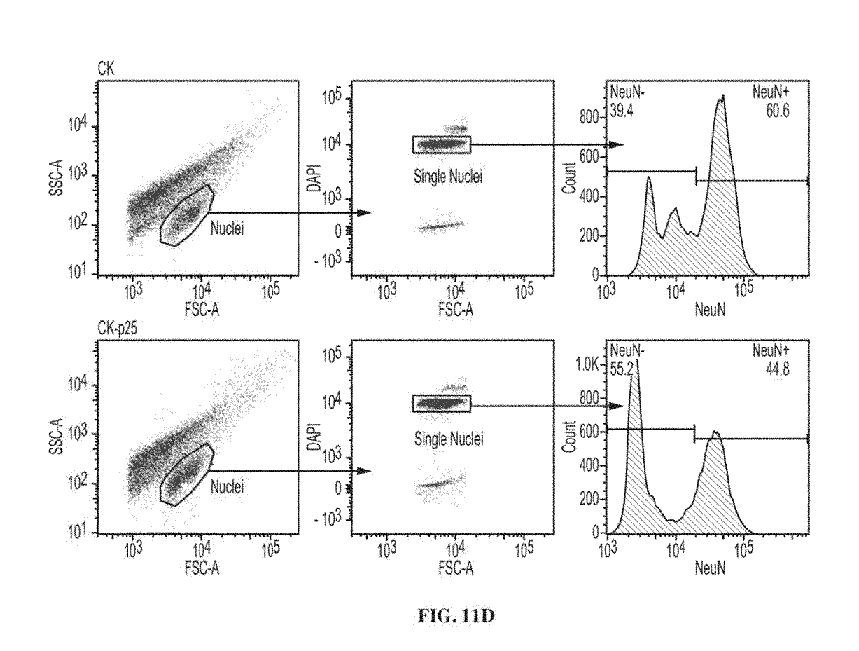

Next, Sp3 levels in CK-p25 mice were examined. The expression of HDAC2 was elevated in the cortex and the hippocampus of the 6-week induced CK-p25 mice (FIGS. 11A-11B). Interestingly, Sp3 protein levels were also elevated in the cortex (FIG. 4A) and hippocampus (FIG. 11B) of the 6-week induced CK-p25 mice. Similarly, the complex of HDAC2 and Sp3, as assessed by co-immunoprecipitation with an anti-HDAC2 antibody, was increased in the CK-p25 mouse (FIGS. 4B and 11C). Importantly, the levels of HDAC2 and Sp3 bound to the promoters of synaptic genes downregulated in 6-week induced CK-p25 mice were assessed. Consistent with the notion that the HDAC2-Sp3 complex antagonizes synaptic gene expression in these mice, increased HDAC2 and Sp3 binding was found at many of these loci in CK-p25 NeuN+ neuronal nuclei compared to the CK control (FIGS. 4C and 4D and 11D).

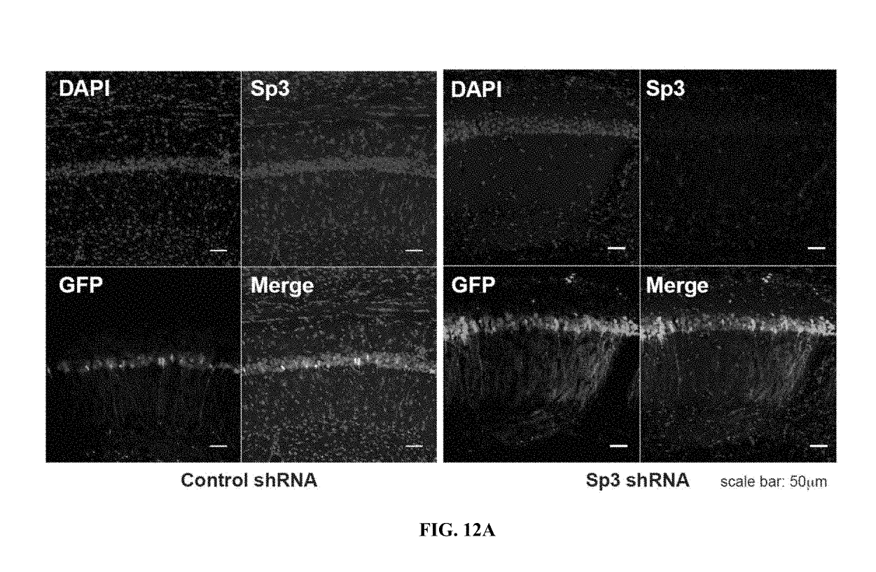

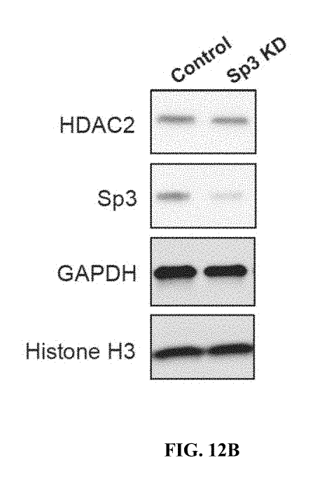

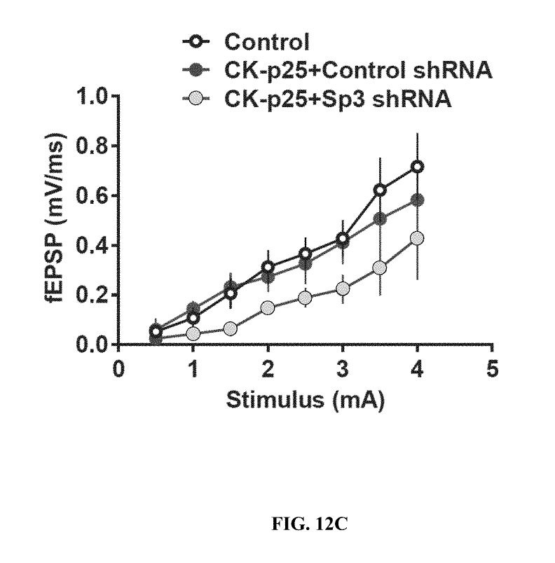

To test the importance of elevated Sp3 levels to AD-related pathology, an shRNA targeting Sp3 in the hippocampus of CK-p25 mice was expressed (FIGS. 12A-12B). Previous experiments showed that expression of an HDAC2 shRNA to normalize HDAC2 levels in CK-p25 mice was sufficient to reverse deficits in long-term synaptic plasticity. While long-term potentiation (LTP) in the CA3-CA1 Schaffer collateral pathway was severely impaired in CK-p25 mice injected with control shRNA, CK-p25 mice injected with Sp3 shRNA showed robust LTP comparable to control mice (FIG. 4E). Sp3 knockdown did not significantly affect basal synaptic transmission in CK-p25 mice (FIG. 12C).

Taken together, these results show that both HDAC2 and Sp3 are up-regulated in CK-p25 model mice and postmortem AD hippocampal tissue. Further, these results demonstrate that, like HDAC2, down-regulation of Sp3 expression ameliorated deficits in synaptic plasticity in CK-p25 mice.

### Example 5: Inhibiting the HDAC2-Sp3 Complex Enhances Synaptic Function

The experimental data provided herein demonstrates that Sp3 plays a key role in the recruitment of HDAC2 to the promoters of synaptic genes and that this mechanism is deregulated in Alzheimer's disease. Unlike HDAC2, HDAC1 does not repress synaptic gene expression and cognitive function although the two proteins share 80% amino acid homology, with the greatest divergence at the carboxyl terminus (C-terminus). Instead, loss of HDAC1 results in double-stranded DNA breaks, aberrant reentry into the cell cycle, and neuronal death. HDAC1 gain-of-function is neuroprotective.

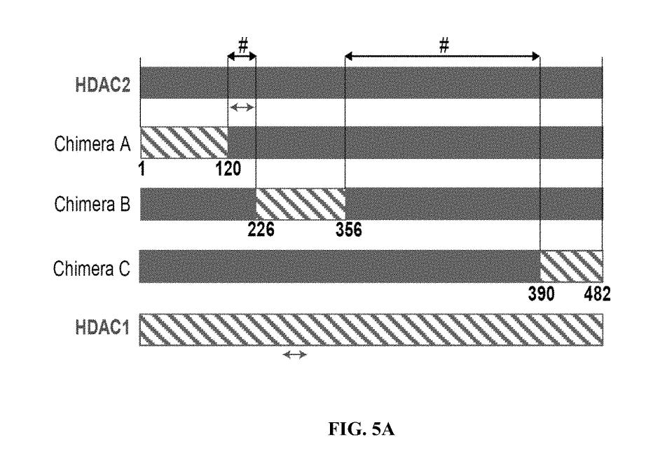

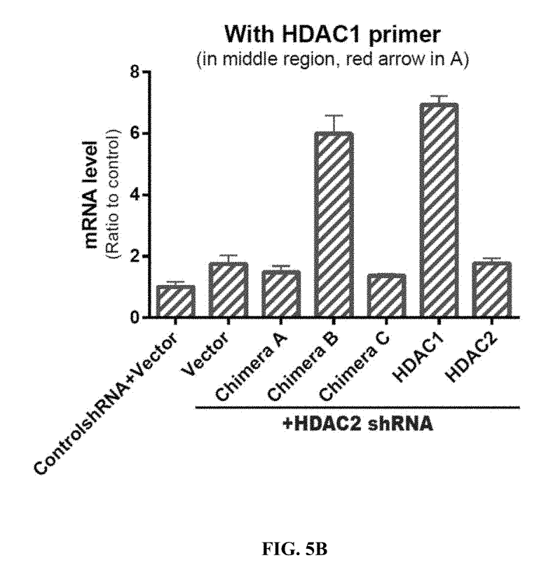

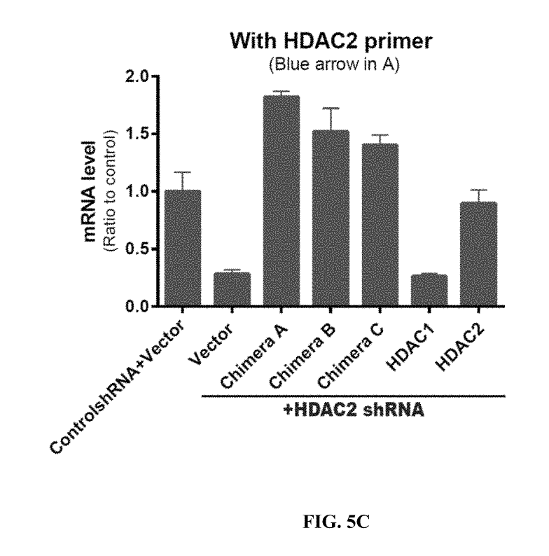

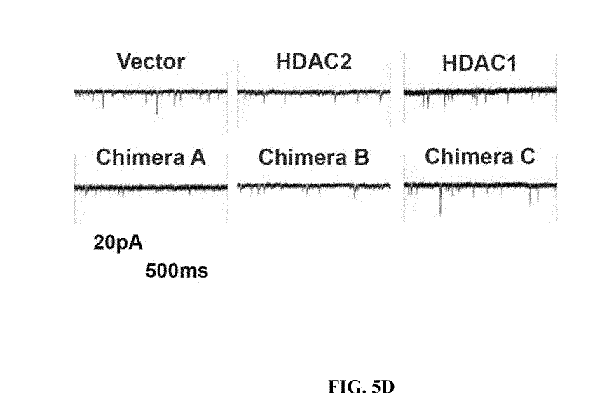

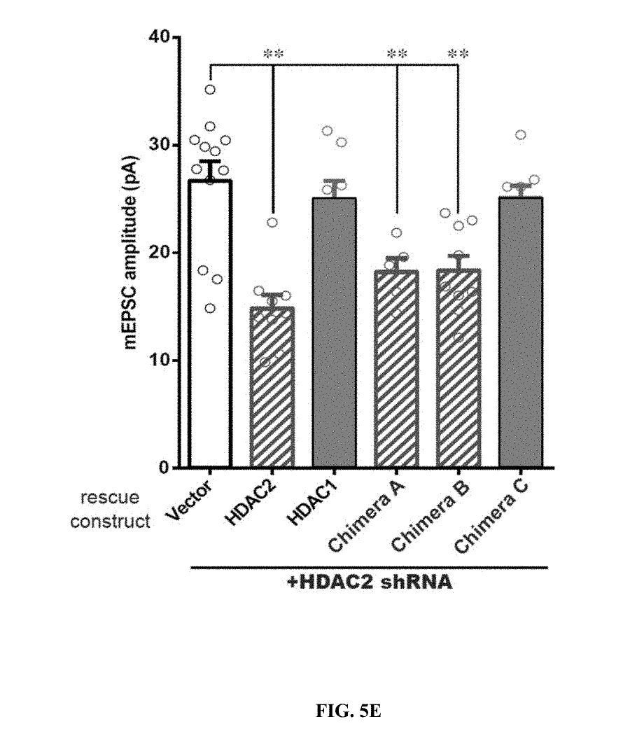

To further characterize the HDAC2-Sp3 interaction, the region of HDAC2 involved in regulating synaptic functions and binding to Sp3 was mapped. Three chimeras of HDAC2 and the closely related HDAC1, each of which contains the highly conserved HDAC2 catalytic domain and nuclear localization signal, were generated (FIG. 5A). For chimera A, the amino terminus of HDAC2 (amino acids 1-121) was replaced with that of HDAC1 (amino acids 1-120). In chimera B, the middle domain of HDAC2 (amino acids 227-357) was replaced with that of HDAC1 (amino acids 226-356). In chimera C, the divergent C-terminus of HDAC2 (amino acids 391-488) was replaced with that of HDAC1 (amino acids 390-482).

Each of these chimeras were expressed in cultured primary neurons, and levels of expression were determined using primers complementary to HDAC1 and HDAC2 (primer binding regions marked with arrows in FIG. 5A). After knockdown of HDAC2 in cultured neurons, only chimera B expressed the middle portion of HDAC1 at the same level as full length HDAC1 (FIG. 5B). Furthermore, chimeras A, B, and C expressed a region of HDAC2 between amino acids 120-226 at similar levels, unlike full-length HDAC1, suggesting that any differential effects seen in subsequent experiments are not due to variable expression of the constructs (FIGS. 5B-5C).

Each construct was then tested for its ability to dampen the increased mEPSCs amplitude caused by HDAC2 knockdown in cultured primary neurons. Notably, expression of full length HDAC1 or chimera C (HDAC2 with the C-terminus of HDAC1) did not counteract the effect of HDAC2 knockdown on mEPSCs (FIGS. 5D-5E). In contrast, chimera A and chimera B, as well as full length HDAC2, did significantly rescue HDAC2 knockdown (FIGS. 5D-5E). These data suggest that the divergent C-terminus of HDAC2 is critical for regulating synaptic function.

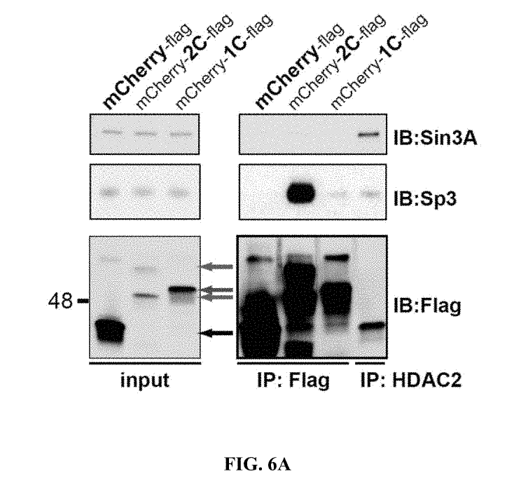

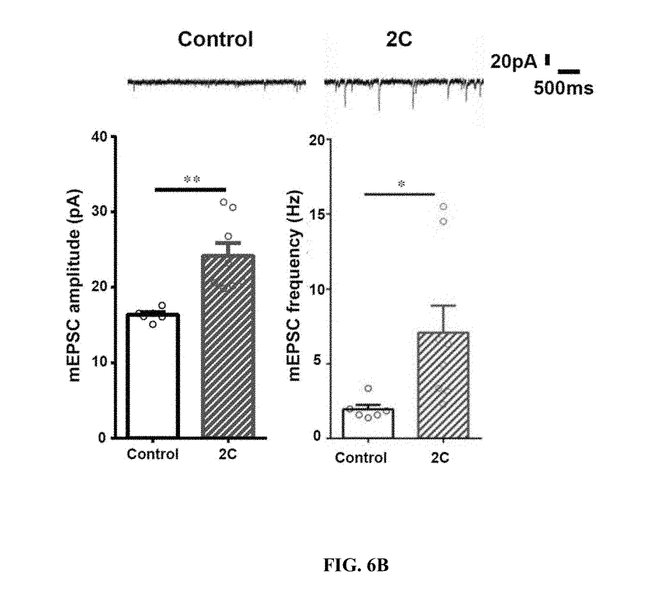

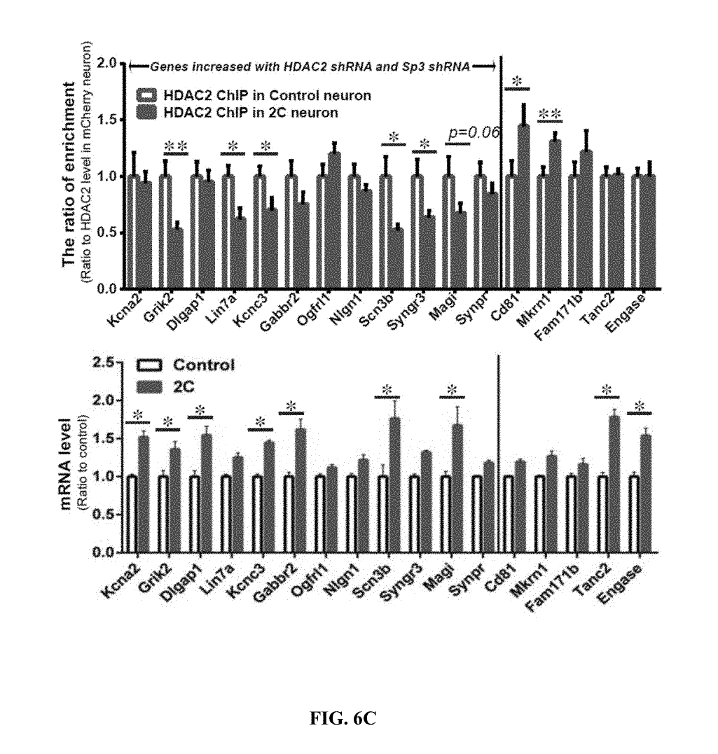

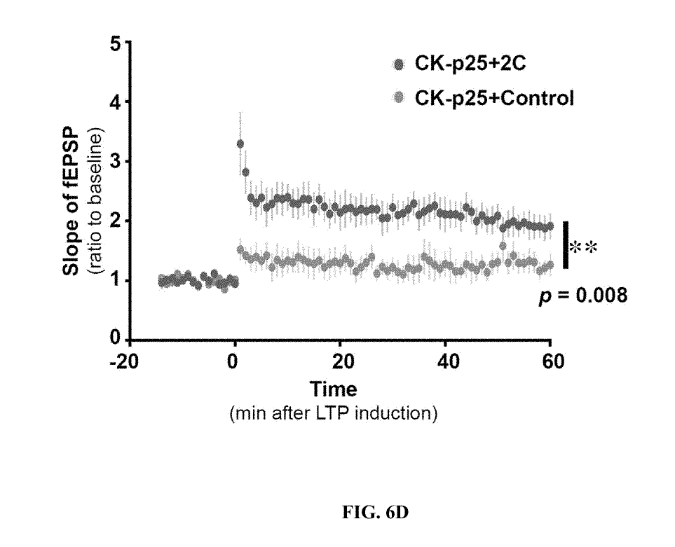

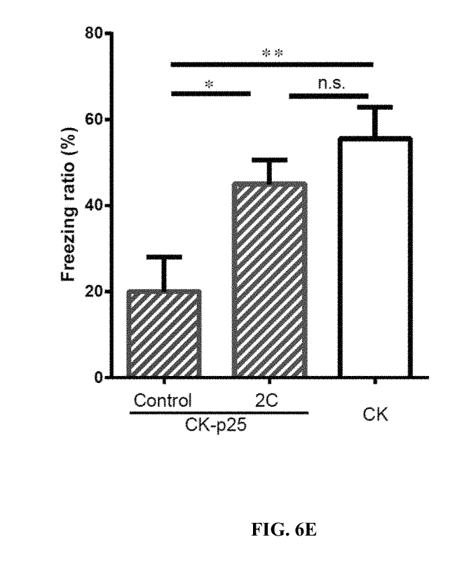

If the divergent C-terminus of HDAC2 alone is capable of binding to Sp3, the HDAC2-Sp3 interaction may potentially be inhibited through over-expression of this domain. To test this, the C-terminal domain of either HDAC2 (termed 2C) or HDAC1 (termed 1C) fused with mCherry, or mCherry alone, was transfected into neuronal N2A cells. Using co-IP experiments it was shown that 2C, but not 1C or mCherry alone, robustly bound to endogenous Sp3 (FIG. 6A). Importantly, binding of 2C to Sin3A, a well characterized partner of the HDAC1/2 complexes that controls cell cycle progression, was not detected. This result suggests that Sin3A binds to a different region of HDAC2.

Next, whether synaptic function was affected by the expression of 2C was examined. Results showed that expression of 2C in cultured primary neurons facilitated mEPSC amplitude and frequency reminiscent of either HDAC2 or Sp3 knockdown (FIG. 6B). Whether recruitment of HDAC2 to synaptic genes was perturbed by expression of 2C, as it was by knockdown of Sp3, was also tested (FIG. 2D). Consistently, HDAC2 enrichment at the promoters of genes involved in synaptic transmission was significantly reduced after the expression of 2C (FIG. 6C). Further, increased expression of the majority of synaptic genes tested after the expression of 2C was observed (FIG. 6D). Together, these data indicated that overexpression of the C-terminal domain of HDAC2 mimics the effects of HDAC2 and Sp3 knockdown on synaptic function, gene expression and HDAC2 localization across DNA, possibly through the eviction of HDAC2 from the relevant genomic loci.

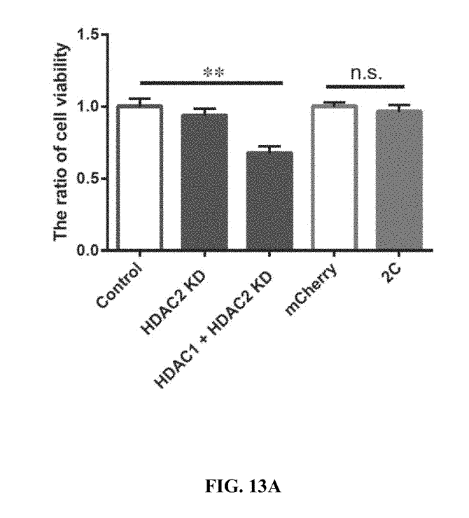

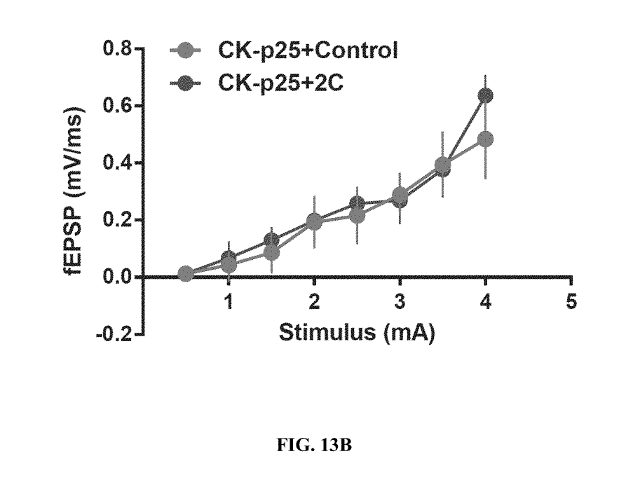

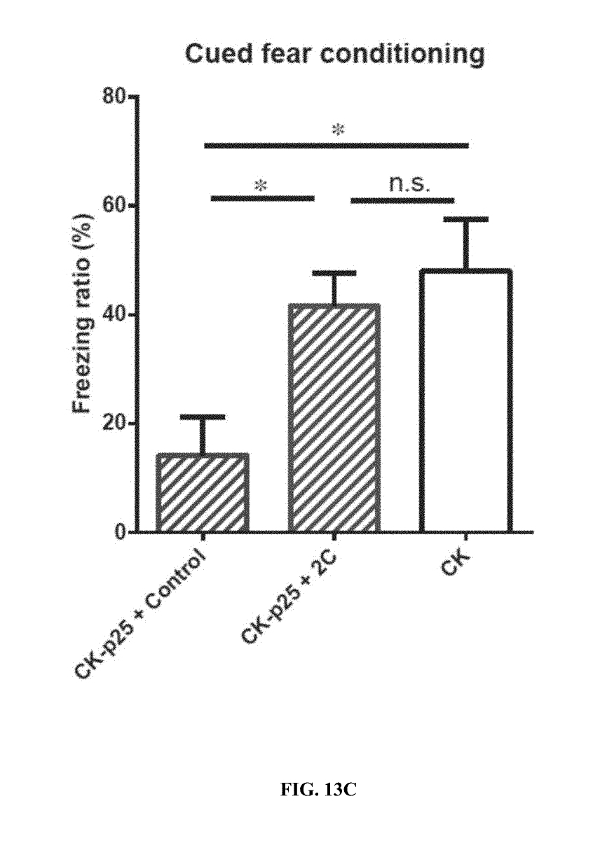

Next, whether inhibition of HDAC2 recruitment to the promoters of synaptic genes via overexpression of 2C affects cell proliferation was examined. Currently available pan-HDAC inhibitors block cell cycle progression, which could elicit undesirable effects. Therefore, whether proliferation of mouse embryonic fibroblasts (MEFs) was affected by overexpression of 2C was tested. While the rate of proliferation in MEFs was significantly decreased by simultaneous knockdown of HDAC1 and HDAC2, no effect of 2C expression on proliferation compared to mCherry controls was observed (FIG. 13A). These results suggest that targeting the C-terminal domain of HDAC2 enables selective manipulation of synaptic function while avoiding deleterious effects on cell proliferation.

As a validation of the therapeutic potential of targeting the HDAC2-Sp3 complex through the expression of 2C, the effects of 2C expression on CA3-CA1 Schaffer collateral LTP and memory function using the CK-p25 model of neurodegeneration was tested. Lenti-viral expression of 2C, but not control virus, in the hippocampus of the CK-p25 mouse had no effect on basal synaptic transmission, but enhanced LTP in these mice (FIGS. 6E and 13B). Hippocampus-dependent memory formation, as evaluated by contextual and cued fear-conditioning assays, is also markedly impaired in the 6-week induced CK-p25 mouse. Importantly, overexpression of 2C in the hippocampus was able to ameliorate both context-dependent and cued fear learning deficits (FIGS. 6F and 13C). Thus, overexpression of 2C can counteract synaptic and cognitive deficits in a mouse model of neurodegeneration. Taken together, our findings indicate that targeting the C-terminus of HDAC2 constitutes a plausible and specific strategy to inhibit the HDAC2-Sp3 complex and treat neurological disorders associated with memory impairment.

### SEQUENCES

### SEQ ID NO: 1—HDAC2 Peptide Inhibitor (Human HDAC2, UniProt ID No.: Q92769, Amino Acids 390-488):

VHEDSGDEDGEDPDKRISIRASDKRIACDEEFSDSEDEGEGGRRNVADHK
KGAKKARIEEDKKETEDKKTDVKEEDKSKDNSGEKTDTKGTKSEQLSNP

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can easily ascertain the essential characteristics of the present disclosure, and without departing from the spirit and scope thereof, can make various changes and modifications of the present disclosure to adapt it to various usages and conditions. Thus, other embodiments are also within the claims.

### EQUIVALENTS AND SCOPE

Those skilled in the art will recognize, or be able to ascertain using no more than routine experimentation, many equivalents to the specific embodiments of the present disclosure described herein. The scope of the present disclosure is not intended to be limited to the above description, but rather is as set forth in the appended claims. In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A method for treating a neurodegenerative disease in a subject, comprising:
administering to the subject an effective amount of a histone deacetylase 2 (HDAC2)/transcription factor Sp3 (Sp3) inhibitor, wherein the HDAC2 inhibitor reduces HDAC2 binding to transcription factor Sp3 (Sp3) to treat the neurodegenerative disease.

2. The method of claim 1, wherein the HDAC2/Sp3 inhibitor is a peptide.

3. The method of claim 2, wherein the peptide HDAC2/Sp3 inhibitor is an anti-HDAC2 antibody.

4. The method of claim 1, wherein the HDAC2/Sp3 inhibitor is a small molecule inhibitor.

5. The method of claim 2, wherein the peptide is about 25-110 amino acids in length.

6. The method of claim 2, wherein the peptide is an amino acid sequence that is at least 80% identical to SEQ ID NO: 1.

7. The method of claim 1, wherein the neurodegenerative disease is selected from the group consisting of MCI (mild cognitive impairment), post-traumatic stress disorder (PTSD), Alzheimer's Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, vascular dementia, progressive supranuclear palsy or cortical basal degeneration.

8. The method of claim 1, wherein the amount of HDAC2/Sp3 inhibitor is effective in reducing synaptic dysfunction.

9. The method of claim 1, wherein the amount of HDAC2/Sp3 inhibitor is effective in reducing histone deacetylation.

10. The method of claim 1, wherein the HDAC2/Sp3 inhibitor is formulated in a pharmaceutical composition, which further comprises a pharmaceutically acceptable carrier.

11. The method of claim 1, further comprising administering to the subject another therapeutic agent.

12. The method of claim 1, wherein the subject is a human patient.

13. A method for treating a neurodegenerative disease in a subject, comprising:
administering to the subject an effective amount of a transcription factor Sp3 (Sp3) expression inhibitor to reduce Sp3 expression levels in the subject in order to treat the neurodegenerative disease.

14. The method of claim 13, wherein the Sp3 expression inhibitor is an antisense oligonucleotide.

15. The method of claim 13, wherein the Sp3 expression inhibitor is an siRNA.

16. A method for treating a neurodegenerative disease in a subject, comprising:
administering to the subject an effective amount of a histone deacetylase 2 (HDAC2) localization inhibitor, wherein the HDAC2 localization inhibitor reduces HDAC2 localization to chromatin to treat the neurodegenerative disease.

17. The method of claim 16, wherein the HDAC2 localization inhibitor is an HDAC2/Sp3 inhibitor.

18. A pharmaceutical composition, comprising a peptide of 20-110 amino acids in length having an amino acid sequence that has at least 80% sequence identity to SEQ ID NO: 1 and a pharmaceutically acceptable carrier.

19. The composition of claim 18, wherein the peptide is about 80-100 amino acids in length.

20. The composition of claim 18, wherein the peptide comprises an amino acid sequence that has at least 85% sequence identity to SEQ ID NO: 1.

21. The composition of claim 18, wherein the peptide comprises an amino acid sequence that has at least 90% sequence identity to SEQ ID NO: 1.

22. The composition of claim 18, wherein the peptide comprises an amino acid sequence that has at least 95% sequence identity to SEQ ID NO: 1.

23. The composition of claim 18, wherein the peptide comprises the amino acid sequence of SEQ ID NO: 1.

24. The composition of claim 18, wherein the peptide consists of the amino acid sequence of SEQ ID NO: 1.

25. The composition of claim 24, wherein the pharmaceutically acceptable carrier is a nanoparticle, intravenous fluid, buffered pharmaceutical solution, cream, emulsion, gel, liposome, or ointment.

26. A peptide of 20-110 amino acids in length having an amino acid sequence that has at least 80% sequence identity to SEQ ID NO: 1 and includes at least one amino acid that is non-naturally occurring in an HDAC2 peptide of SEQ ID NO: 1.

27. The peptide of claim 18, wherein the peptide comprises an amino acid sequence that has at least 85% sequence identity to SEQ ID NO: 1.

28. The peptide of claim 18, wherein the peptide comprises an amino acid sequence that has at least 90% sequence identity to SEQ ID NO: 1.

29. The peptide of claim 18, wherein the peptide comprises an amino acid sequence that has at least 95% sequence identity to SEQ ID NO: 1.

30. A pharmaceutical composition for treating a neurodegenerative disease in a subject, the composition comprising (i) an effective amount of a histone deacetylase 2 (HDAC2)/transcription factor Sp3 (Sp3) inhibitor; and (ii) a pharmaceutically acceptable carrier.
